(19) United States
(12) Patent Application Publication
(10) Pub. No.: US 2014/0154735 A1
(43) Pub. Date: Jun. 5, 2014

(54) TUMOUR CELL AND TISSUE CULTURE

(71) Applicants: Lars Sundstrom, Bristol (GB); Thelma Biggs, Southampton (GB); Janet Foreman, Devon (GB)

(72) Inventors: Lars Sundstrom, Bristol (GB); Thelma Biggs, Southampton (GB); Janet Foreman, Devon (GB)

(73) Assignee: CAPSANT NEUROTECHNOLOGIES LTD., London (GB)

(21) Appl. No.: 13/978,209

(22) PCT Filed: Jan. 6, 2012
(86) PCT No.: PCT/EP2012/050194

§ 371 Date: Jan. 28, 2014

(30) Foreign Application Priority Data
Jan. 6, 2011 (GB) 1100180.7

(51) Int. Cl. G01N 33/50

(52) U.S. Cl. CPC G01N 33/5014
USPC 435/32; 435/29; 435/347; 435/373

(57) ABSTRACT

The present invention relates to in vitro three-dimensional organotypic cell co-culture. Cultures of the invention comprise tumour cells three-dimensionally disposed within a matrix of matrix cells which are distinct from the tumour cells, wherein the co-culture does not comprise a basement membrane. The cultures are useful for the study of cancers, for testing anti-tumour agent efficacy, and for high-throughput screening of candidate drugs.

The present invention relates to in vitro tumour cell and tissue culture. In particular, the present invention relates to co-cultures, i.e., mixed cell cultures, comprising tumour cells and cells that are distinct from said tumour cells. The co-cultures of the invention are three-dimensional (3D) organotypic cultures. The invention relates to the formation of co-cultures by aggregation of dissociated cells. The tumour cells in the co-cultures of the invention may be derived from any source of tumour cells, including e.g. primary tumour tissue or tumour cell lines. Methods for preparing the cultures of the invention are also provided. Cultures of the invention are useful for the study of cancer, cancer therapy and cancer diagnostics. For example, cultures of the invention are useful in methods for screening for anti-cancer agents and for testing the efficacy of anti-cancer agents. The methods and cultures of the invention are well suited to applications in a high-throughput format.

Despite aggressive treatment protocols, the prognosis for many types of cancer is still extremely poor. Identification of new therapeutic regimens is urgently needed. However, a major challenge for the pharmaceutical industry remains the development of relevant model systems to profile compounds. For example, although glioblastoma multiforme (GBM) is the most common and most aggressive type of primary brain tumour in humans, no satisfactory model system for GMB is available to date. For this and other important human cancers, there is an urgent need to develop novel approaches to cultivating tumour cells, in order to provide tumour models which allow more faithful replication of relevant physiological conditions within tumours and improve the predictive value of screens for anti-cancer agents.

According to the currently established practice, immortalised tumour cell lines are used extensively to study the mechanisms of how various cancers develop and to test the efficacy of new treatments. Initial screening of compounds of potential relevance in anti-cancer chemotherapy is currently based on their ability to kill or inhibit the proliferation of 60 established and well characterised tumour cell lines from a “certified” NCI panel in two dimensional (2D) culture conditions (Damia 2009). The 2D mono-cultures have a single tumour cell type and are grown using standard operating procedures with around 20,000 cells per well in a 96 well plate and then pre-incubated in the absence of drug for 24 hours. Test agents are then added and incubated for 48 hours. At the end of this incubation, sulforhodamine B is used to assay the protein levels of the cells. This standardised assay has been used to screen more than 85,000 compounds to date. Of those that were effective in 2D cultures only a small proportion has been successful in the clinic. A large majority of novel anti-cancer agents fail in the clinic despite evidence of anti-tumour activity in conventional in vitro assays.

In vivo models for growing tumours have been developed (Rygaard and Povlsen 1969; Kelland L. R 2004). However, these models are expensive, time consuming, require live animal experiments. Often they are subcutaneous and thus do not allow the tumour to develop in the organ that it was derived from, despite the in vivo setting. Such models are also not suitable for use in methods in a high-throughput format.

Beaupain, 1999 and Starzec, 2003 relate to co-cultures of tumour cells with other cells. In these methods, the tumours are encouraged to form nodules in semisolid media. The nodules form from cell to cell contacts between the cancer cells, and as they form the other cell types are incorporated into the nodules. This is not representative of an in vivo situation, in which tumours form within a matrix of existing non-tumour cells. The centre of the nodules also tends to become anoxic as they grow. Uses of these cultures are therefore also limited because they have to be reformed too frequently. Moreover, different cell types may be incorporated on each passage, which leads to an undesirable level of variability between these cultures.

Ridky et al., (Nature Medicine (2010) 16(12):1450-1455) have described cultures of certain types of epithelial tumour cells, in which the recombinantly transformed epithelial cells are cultured upon one side of a basement membrane preparation, with a layer of fibroblasts on the other side of the basement membrane preparation. This model is limited to specific epithelial situations, relies upon recombinant transformation of the epithelial cells and upon a particular spatial disposition of transformed cells and fibroblasts, on either side of a basement membrane preparation.

The inventors have developed novel, versatile methods of culturing tumour cells which provide a broad range of physiologically relevant organotypic tumour models. The tumour cell cultures of the invention have surprising properties which overcome many of the shortcomings of currently available cell cultures. The organotypic tumour cell cultures of the invention provide a reliable, reproducible, rapid and inexpensive alternative to in vivo experiments.

### DESCRIPTION OF THE INVENTION

### Summary of the Invention

The present invention provides tumour cell cultures, more particularly an in vitro three-dimensional organotypic cell co-culture comprising tumour cells three-dimensionally disposed within a matrix of matrix cells which are distinct from the tumour cells.

A co-culture according to the invention does not comprise an slice or piece of an organ or solid tissue, or any other sample of adherent cells from a solid tissue. Rather, a co-culture of the invention is formed by aggregation of dissociated cells, i.e., from cells in suspension, in dissociated form. If the cells from which cultures are formed originate from a source comprising adherent cells (e.g. an adherent cell culture or a tissue or an organ sample or slice), the originally adherent (aggregated) cells will have been dissociated (disaggregated) prior to formation of the co-culture of the invention. According to the invention, the dissociated cells aggregate during formation of the co-culture. In particular, the cells aggregate following concentration and/or compaction of the co-cultured cells from a cell suspension. Co-cultures of the invention may thus also be termed “aggregated” or “re-aggregated” co-cultures.

A co-culture of the invention may comprise a semi-permeable support and a liquid culture medium, wherein
a) the co-culture is disposed upon a first surface of the support facing a gaseous phase;
b) a second surface of the support faces and is in contact with the liquid culture medium; and
c) the liquid culture medium is retained in contact with the support and with the culture by virtue of surface tension and/or capillarity.

Thus, the culture may be situated on the support at a gas-liquid interface. The gaseous phase may be any gaseous phase or composition commonly used for cell culture, e.g. air. Thus the gas-liquid interface may be an air-liquid interface.

The invention also provides a method of preparing a three-dimensional organotypic cell co-culture comprising the steps of
a) providing a first cell suspension comprising tumour cells and a second cell suspension comprising matrix cells which are distinct from the tumour cells;
b) combining the first and second cell suspensions to provide mixed cells suspended in a liquid suspension medium;
c) concentrating and/or compacting the mixed cells;
d) incubating the mixed cells obtained from step (c) on a first surface of a semi-permeable support facing a gaseous phase, wherein during the incubation a second surface of the support faces and is in contact with a liquid culture medium, and wherein the liquid is retained in contact with the support and the mixed cells by virtue of surface tension and/or capillary action.

In preferred embodiments of the co-cultures and methods of the invention, the second surface of the support is contralateral to the first surface.

### PROPERTIES AND ADVANTAGES OF THE INVENTION

The matrix cells form the organotypic environment in which the tumour cells grow. The term “organotypic culture” indicates that cells in the culture associate in a way that as closely as possible replicates the biochemical and physiological properties of the organ from which the cells are derived. In a “co-culture”, cells of at least two distinct types are cultured together. Co-cultured cells may be derived from different sources but can still associate together to replicate an in vivo situation. Co-cultures have properties that are derived from all cell types and from the interaction between the cells within the co-culture.

The inventors have surprisingly found that a 3D organotypic culture of matrix cells can grow and sustain tumour cells within the culture, so as to provide an in vitro 3D organotypic tumour cell culture according to the invention. The scaffold or matrix for tumour cell growth is provided primarily by the non-tumour cells themselves during culture. Surprisingly, both the dissociated matrix cells and the dissociated tumour cells, once mixed according to the methods of the invention, reorganise to form the organotypic co-cultures of the invention. Such formation of such organotypic tumour cell cultures has not previously been shown.

An advantage of the co-cultures of the present invention is that they allow interaction of tumour cells with other cell types that are relevant to in vivo tumour growth and pathology. The matrix cells of the invention provide a physiologically relevant environment for the tumour cells. Compacted matrix cells spontaneously reorganize into a complex 3D functional parenchyma over time. For example, compacted brain cells will lead to the formation of a tissue-like structure, in which much of the appropriate synaptic circuitry, physiology and neurotransmitter receptor distribution of the intact central nervous system region are present. The inventors have found that functional activities of neurons in the culture are similar to their counterparts in brain and in organotypic slice cultures.

Thus, unlike previous models, especially 2D cultures, the co-cultures of the invention allow for a physiological, three-dimensional network of cell to cell interactions. The network is, moreover, not limited to interactions between tumour cells. In vivo, such interactions between tumour cells and surrounding non-tumour cells can be critical for various reasons.

The co-cultures of the invention allow for signalling between various cells that contribute to the physiological environment of the tumour. This is particularly advantageous, as such signals, including growth factors, cytokines, chemokines and matrix degrading enzymes, may have either a positive or negative effect on the signalling pathways that lead to cell death and are thus relevant therapeutic targets.

The three-dimensional environment of the cells in the co-cultures of the invention allows for the natural formation of an extracellular matrix. This is advantageous because the extracellular matrix is an important regulator of cellular behaviour and responses to anti-cancer agents. Tumour cells grown in such a three-dimensional environment may also have morphologies that more closely resemble that observed in the clinic than cells grown in a 2D monolayer. In conventional 2D cultures, or cultures lacking a three diminsional network of different cell types, the extracellular matrix may be absent or different from that found in vivo.

An extracellular matrix that is naturally formed by the co-cultured cells according to the present invention is superior to artificial or simplified protein matrices or scaffolds that may be added to cell cultures. Such exogenous matrices or scaffolds can only provide imperfect substitutes for in vivo tissue matrices. Collagen alone, for example, produces only a loose network of fibres whereas connective tissue has a heterogeneous structure of pore sizes and fibre thickness. Methods employing matrix proteins generally suffer from disadvantages of high cost and variability, and time consuming procedures for preparing the matrix. Growth upon matrices of cell extracts also have the disadvantage that the extracts can be variable and do not provide the cell to cell interactions resembling those that are seen in vivo. These disadvantages are all avoided by the present invention, in which the co-cultured cells provide the appropriate matrix themselves.

The invention provides methods of culturing tumour cells in a physiologically relevant environment that are significantly faster, cheaper, and more flexible that culture of entire organs, or organotypic culture of organ and tissue slices, which has to date been the only satisfactory way of closely replicating in vivo physiological conditions. Moreover, the methods of the invention are much less limited by the availability of materials than organ and tissue slice culture.

The close three-dimensional packing of the cells that can be achieved in the co-cultures of the invention is also significant because potential anti-cancer agents applied to the culture are required to penetrate a closely packed three-dimensional parenchyma, just as in the in vivo environment.

The co-culture model of the present invention has been shown faithfully to replicate important aspects of the way that tumours behave in vivo. For example, the tumour cells migrate through the surrounding cell matrix. The inventors have also surprisingly found that tumour cells form tumour-like aggregates within the organotypic co-culture, closely resembling a relevant in vivo situation, rather than growing in a single monolayer as they do in a 2 D system. Thus, importantly, the inventors have shown that it is possible to develop tumour-like structures in an in vitro organotypic co-culture, within a matrix derived from tissues from which the tumours are derived in vivo.

Certain cells from the matrix or stroma surrounding the tumour cells may act to protect the tumour cells against anti-cancer agents. The co-cultures of the invention can replicate such influences of other cells and the cellular environment on tumours during drug treatments. The co-culture of the invention thus offers the advantage of allowing investigation of compounds whose activity might be affected by the surrounding cell matrix. An example relevant to, e.g., brain tumours is the protective effect of astrocytes which works in some part to negate the efficacy of Taxol as an anti-tumour compound. In skin, macrophages may confer a protective effect upon tumour cells. Such cells may be incorporated in the co-cultures of the invention as matrix cells.

The methods and co-cultures of the present invention are adaptable to many different cell types, both different tumour types to those examples described here and to different matrix cells types and matrix tissues. The methods and co-cultures of the invention may in principle be used to culture any type of tumour cell, and can be adapted to model any tissue type. They offer the flexibility for given tumour cells to be co-cultured with a variety of other cell types (referred to herein as matrix cells). The invention allows any cells of interest to be incorporated in the co-cultures alongside the tumour cells. The composition and properties of the matrix in which the tumour cells grow can be purposively selected and controlled. The co-cultures of the invention also allow the replication and investigation of further factors which may influence the properties of tumours and the efficacy of anticancer agents in vivo, and which may arise due to the presence of non-tumour cells and their interactions with tumour cells. One example is the inter and intracellular pH which may affect the efficacy of some compounds, for example TMZ (temozolamide). Another factor is oxygen penetration and gas exchange, which may often be restricted in tissue cultures compared to tissues in vivo. However, the invention allows for excellent oxygen penetration and gas exchange, as the co-cultures according to the invention are situated at a gas-liquid interface.

As the effects of interactions of tumour cells with their environment can thus be taken into account and studied in a defined manner, the co-cultures of the invention allow drug efficacy data to be obtained that are more relevant to the in vivo situation. The invention thus also opens the door to a better understanding of the factors influencing drug efficacy.

The genotype of tumour cells and matrix cells may vary from patient to patient, and the co-cultures of the invention also provide the flexibility to allow the effects of this variation to be studied in personalised assays.

The presence of both tumour cells and non-tumour cells of an investigator's choice in the co-cultures of the invention also allows both the efficacy and toxicity of candidate anti-cancer agents to be investigated simultaneously.

Cultures according to the invention can display organotypic features for weeks or months. Thus, the co-cultures according to the invention may survive, or may display organotypic features, e.g. for at least 6, at least 7, at least 10, at least 14, at least 17 or at least 21 days. In one example, the inventors cultured for 5 weeks. Thus, the co-cultures according to the invention may survive, or may display organotypic features, e.g., for 6-56 days, e.g. for 7-, 8-, 9-, 10-, 14-, 21-, 28-, 35-, 42-, 49-56 days, e.g., for 7-49 or 14-49 days, e.g. for 28-42 days. The person skilled in the art will appreciate that the length of time a culture survives is may also depend on the aggressiveness of the tumour cells used.

As the co-cultures and methods of the invention do not require particular extracellular protein scaffolds, which are laborious and time consuming to prepare, multiple organotypic co-cultures of the invention may be prepared in parallel, on a large scale. The co-cultures of the invention are easy to produce and maintain, in a reproducible manner. They are therefore are ideal for use in high-throughput formats. This applies to all methods disclosed herein, e.g., screening methods for anti-cancer drug candidates.

Screening for drugs using the co-culture model of the present invention has significant financial benefits, as it can be recognised early on in the development cycle which compounds are likely to be successful in the clinic. The robustness and ease of manipulation of the present model allows such assessments to be made more quickly than previously possible. Compounds that have previously failed in the 2D models of tumour monocultures can be retested for efficacy in the 3D co-culture of the present invention where a proper assessment can be made of how such drugs behave in a model that more closely resembles an in vivo situation. It is also possible to test combinations of new and/or established drugs simply and effectively to establish if some combinations of treatments have synergistic effects on drug efficacy.

Independence from Extracellular Biological Scaffolds and Matrices

The co-cultures of the invention are prepared and grown upon one surface of a semi-permeable support, however, as mentioned above, the methods and co-cultures of the invention do not require exogenous scaffold or extracellular matrix protein.

According to preferred embodiments, the co-cultures and methods of the invention thus do not comprise or involve the use of an exogenous or pre-formed acellular protein scaffold, such as an acellular scaffold comprising collagen. Examples of such a pre-formed or exogenous acellular protein scaffold include a basement membrane, or a scaffold comprising or derived from basement membrane (e.g. a devitalized dermis comprising a basement membrane). As the person skilled in the art will appreciate, a basement membrane is a sheet of extracellular fibres. In a physiological context it underlies epithelia or endothelia. An intact basement membrane is composed of two lamina, the basal lamina and the reticular lamina. The basal lamina comprises type VII collagen. The reticular lamina comprises microfibrils (fibrillin).

In preferred embodiments of the invention, the co-culture does not comprise a basement membrane or derivative, component or variant thereof. In other preferred embodiments, the co-culture does not comprise any pre-formed acellular protein scaffold.

Preferably, in the methods of the invention, no extracellular scaffold protein is added to any of the first or second cell suspensions or the mixture thereof (the suspension of mixed cells). Preferably, in the methods of the invention, no extracellular scaffold protein is added to the co-culture during its preparation, except that in certain embodiments the semi-permeable support may be coated with a material that facilitates adhesion of the co-culture to the support. Likewise, co-cultures of the invention preferably do not comprise any pre-formed acellular protein scaffold, except that in certain embodiments the semi-permeable support may be coated with a material that facilitates adhesion of the co-culture to the support. In certain embodiments of the invention, the semi-permeable support may be coated with a material that facilitates adhesion of the co-culture to the support, but the co-culture does not comprise a basement membrane or any derivative, component or variant thereof. The coating material that facilitates adhesion of the co-culture to the support may be, e.g., selected from collagen, laminin, fibronectin or matrigel. The semi-permeable support itself does not comprise extracellular scaffold protein.

Preferably, the only extracellular matrix proteins contained in the matrix surrounding the tumour cells of the co-culture are extracellular matrix proteins that may be secreted by the cells of the co-culture. Preferably, the culture does not comprise a natural or artificial basement membrane, or any derivative of, or acellular protein scaffold from, a basement membrane.

Herein, all statements (in particular relating to the absence of e.g. exogenous extracellular matrix proteins or acellular protein scaffolds) are made notwithstanding that the semi-permeable support may be coated with a material that facilitates adhesion of the co-culture to the support, such as collagen, laminin, fibronectin or matrigel.

In certain embodiments, the methods and cultures of the invention can incorporate any of the components as mentioned above, such as exogenous extracellular matrix proteins or acellular protein scaffolds, if desired, in addition to any coating of the semi-permeable support.

### Matrix and Tumour Cells in the Co-Cultures of the Invention

According to the invention, the matrix cells provide a matrix in which the tumour cells are initially evenly dispersed. According to the invention, the tumour cells are thus three dimensionally disposed within the matrix of matrix cells. The tumour cells are cultured within the matrix. The tumour cells may in some embodiments migrate through the matrix.

Preferably, the tumour cells form tumour-like aggregates. Such aggregates are generally formed within the matrix, e.g., three-dimensionally disposed within the matrix. As a person skilled in the art would appreciate, tumour-like aggregates may for example comprise at least 5, at least 10, at least 15, at least 20, at least 30, or at least 50 tumour cells. For example, the tumour cells may be present in the form of aggregates of at least 10 cells. In other embodiments, the tumour cells may be present in the form of aggregates of at least 15 cells.

In another embodiment, the tumour cells proliferate in the cell matrix but do not aggregate. Depending on the type of tumour cell used the cells may form aggregates or be dispersed throughout the cell matrix. This may also be affected by the type of cells used to form the cell matrix. In certain embodiments, the tumour cells do not form aggregates but rather migrate to the periphery of the cellular matrix. This has been observed, e.g., for mammalian ovarian cancer cells grown in rat brain cells.

### Cells Used in the Methods and Co-Cultures of the Invention

### General

The organotypic co-cultures of the invention can be prepared from a wide variety of cells, from a wide variety of organs and tissues. The nature of the cells that are used in the process will depend on the organotypic tumour co-culture that is desired.

The cells used in the methods to prepare the co-cultures are dissociated cells. That is, both the tumour cells and the matrix cells are dissociated prior to formation of the culture. “Dissociated” means that the cells are not aggregated, not subject to intercellular adhesion, and are individualised in suspension. The cells used in the methods to prepare the co-cultures may, e.g., be dissociated primary cells or dissociated cells from tissue culture, e.g., cell lines, e.g., tumour cell lines. “Primary cells” refers to cells isolated directly from an organ or tissue of an organism.

Cells for use in the co-cultures of the invention (as tumour and/or as matrix cells) may be obtained from a particular region of an organ. For example, where the source organ is brain, the cells may be obtained from the hippocampus or from the cortex. Where the organ is heart, the cells may be obtained from the myocardium.

Preferably, the cells used in the present invention (matrix and/or tumour cells) are mammalian. In preferred embodiments, the cells are human.

The cells used in the methods and co-cultures of the invention (e.g., the tumour cells) may be stem cells. Herein, the term “stem cells” refers to cells that are at least pluripotent and can thus be induced to differentiate into more than one lineage. Some stem cells are capable of differentiation into multiple cell lineages. Stem cells may, e.g., be adult stem cells, embryonic germ cells (e.g. from a non-human mammal, e.g., non-human primate, rodent or mouse), or embryonic stem cells (e.g. from a non-human mammal, e.g., non-human primate, rodent or mouse). Embryonic stem cells can in principle differentiate into any cell type. Where embryonic stem cells are used, they can be human embryonic stem cells provided the legal and ethical issues are addressed. Embryonic stem cells, e.g., human embryonic stem cells, may be obtained from existing embryonic stem cell lines, e.g., cell lines listed on the NIH Human Embryonic Stem Cell Registry. Stem cells have also been found in tumours. Such tumour stem cells (also termed cancer stem cells) can be isolated and used as the tumour cells to create organotypic co-cultures according to the invention.

The cells may be genetically engineered, e.g., they may be derived from a transgenic animal, e.g., from a transgenic non-human mammal.

### Matrix Cells

According to the invention, the matrix cells comprise cells which are distinct from the tumour cells of the invention. The matrix cells comprise at least one cell type which is distinct from the tumour cells of the invention.

The matrix cells may consist of a single cell type, or may comprise more than one cell type. E.g., the matrix cells may comprise at least 2 different cell types, or 3, 4, 5, 6, 7, 8, 9 or 10 different cell types. In certain preferred embodiments, matrix cells are of a cell type that is present in the tissue type of the tumour. In other embodiments (e.g., in order to investigate tumour invasiveness or the influence of different surrounding cell matrices upon tumour cells), the matrix and tumour cells may be of different origins or representative of different tissue types or organs. In principle, the matrix cells may contain, or the composition of the matrix cells may be representative of any or all the cell types that are present in a tissue type or organ of interest (e.g., the tissue type of the tumour cells).

The matrix cells may comprise cells derived from primary tissue or from cell or tissue cultures or cell lines. The matrix cells may be non-cancerous. The matrix cells may also be cancerous or may comprise immortalised cells. That is, the matrix cells may in some embodiments also comprise cancerous cells or tumour cells, provided the matrix cells are distinct from the tumour cells of the co-culture of the invention. The matrix cells preferably comprise non-cancerous cells, i.e. non-tumour cells. In preferred embodiments, matrix cells consist of non-cancerous cells.

The matrix cells may be of any cell, tissue or organ type. The matrix cells may comprise stem cells, primary cells, stromal cells, or cells derived from a source selected from animal tissue, human biopsy, human cadavaric tissue, human tissue derived cell lines and human stem cells, the central nervous system, brain, spinal cord, bone marrow, blood (e.g. monocytes), spleen, retina, thymus, heart, mammary glands, liver, pancreas, thyroid, skeletal muscle, kidney, lung, intestine, ovary, bladder, testis, uterus and connective tissue.

### Tumour Cells

The tumour cells may likewise be may be of any cell, tissue or organ type. They may, in particular, be of any of the types listed above for the matrix cells. They may be derived from cell lines, or from primary cancer cells. They may also be cancer stem cells. Preferably, the tumour cells may be selected or derived from tumours of stem cells, pancreas, blood, cervical, colon, intestine, kidney, brain, mammary gland, ovary, prostate, skin, liver, lung and spleen.

### ASPECTS OF THE METHODS OF THE INVENTION

Preferably, during the incubation of the mixed cells upon a surface of a semi-permeable support facing a gaseous phase in step (d) of the method of the invention, the tumour cells grow three-dimensionally disposed within a matrix formed by the matrix cells.

According to the methods of the invention, the cells in the first and second cell suspensions are dissociated cells.

Methods of the invention may comprise steps to provide the first and/or second cell suspensions, said steps comprising disassociating (i.e., disaggregating) cells from a solid tissue source. That is, the first and/or second cell suspensions comprise dissociated (i.e., disaggregated) cells. A solid tissue source may, e.g., be a tissue sample obtained from an organism (i.e., a subject), or may be a solid tissue culture.

Methods for isolating dissociated cells from organs are known in the art. The dissociated cells may be isolated from the organ of interest by mechanical or enzymatic dissociation of tissue, or both. For example the dissociated cells may be obtained by dissociation of the organ using the proteolytic enzyme trypsin 0.25% (w/w) in Hank's Balanced Salt Solution (HBSS) without calcium and magnesium. After the addition of trypsin inhibitor to stop the enzymatic dissociation, the cells may be incubated briefly in suspension to allow undissociated cells to fall to the bottom, leaving the dissociated cells in suspension. The cells may also be dissociated mechanically by trituration, repeated aspiration of the tissue pieces through smooth glass pipettes of decreasing bore size the resultant cells can be separated from the debris through a cell strainer and the resultant cell suspension prepared as for enzyme dissociated tissue.

The tumour cell suspension may also be prepared by culturing a desired tumour cell line in flasks and then harvesting the cells either by trypsinisation for adherent cells or decanting for non-adherent cells. Tumour cells from biopsy material may also be used. Here, the cells may be dispersed by trypsinisation and/or trituration. The removed cells are then washed with the suspension media and then counted and compacted by centrifugation. The resultant pellet of tumour cells is then resuspended in suspension media to the same concentration as the matrix cells to allow simple calculation of the cell ratios.

Living cells do not behave as rigid bodies, but as substantially incompressible bodies. They deform and adapt their surfaces to adhere to one another, but their volume remains substantially constant unless they lose fluid. If sufficient compaction force is applied, 100% close packing will be achieved, i.e. the cells will be fully close packed with all cell membranes in contact with those of a neighbouring cell. 100% close packing is the maximum number of cells per unit volume for a given cell size without pressurizing them to the point where they lose fluid. It has been found that a culture of cells functions as an organotypic culture if the average degree of close packing between the elements that comprise that culture is at least approximately 10%, more preferably 5%. The dissociated cells may be compacted by any known means in order to achieve the preferred degree of close packing. The compaction force applied should be sufficient to bring the cells into close contact to reach the desired level of close packing without causing cell damage. Preferably, the compaction force applied to the dissociated cells is less than 2×10⁻³ dyne per cell to avoid damage to the cells, more preferably between 10⁻⁵ dyne per cell and 5×10⁻⁴ dyne per cell.

To prepare the cell suspension, the cells may be compacted by gravitational, hydrodynamic or hydrostatic forces. Preferably, the cells are compacted by a gravitational field applied by centrifugation. The cells may also be compacted by aspiration. A combination of more than one mechanism of compaction may also be used. For example, the cells may be compacted by centrifugation and then subsequently by aspiration. The cells may be allowed to settle by gravity also. Centrifugation is preferred as it is rapid and there is less risk of cell damage due to anoxic conditions in the media. The resultant pellet is isolated from the supernatant, for example, by decanting the supernatant. The pellet may be resuspended in a suitable liquid culture medium to a desired cell concentration. The tumour cell suspension can be prepared in a similar manner using standard procedures. The cells in both the cultures can then be counted so that the mixture can be prepared with the desired ratio of non-tumour cells to tumour cells.

Where the cells are compacted by centrifugation, cells in suspension are preferably compacted by centrifugation at 100-500 g, which applies a force in the preferred range of between 10⁻⁵ dyne per cell and 5×10⁻⁴ dyne per cell.

The cells may be compacted separately. The pellets may be resuspended in a medium separately to provide a desired ratio of cells. The tumour cell suspension (the first cell suspension) matrix and the matrix cell suspension (the second cell suspension) may then be combined in amounts as required and placed on a support. Alternatively, the mixed cell suspension may be centrifuged slightly to reaggregate to a specific density prior to placing on the support.

The ratio of matrix cells to tumour cells prior to incubation is not particularly limited, and the skilled person would be readily able to determine suitable cell ratios. In most embodiments, prior to incubation the mixed cell suspension comprises cells in a ratio of 99.999% to 0.001% matrix cells to 0.001% to 99.999% tumour cells. Alternatively, the ratios of matrix cells to tumour cells in most embodiments may be expressed as ranging from 10⁴ to 10⁻⁴. Preferably, the ratio of cells is between 99.5% to 70% matrix cells and 0.5% to 30% tumour cells, 99% to 80% matrix cells and 1% to 20% tumour cells, 99% to 90% matrix cells and 1% to 10% tumour cells, or 98% to 92% matrix cells and 2% to 8% tumour cells. Alternatively, preferred ratios of matrix cells to tumour cells may be expressed as ranging from 200:1 to 2:1; or from 100:1 to 10:1; or from 50:1 to 10:1. According to preferred embodiments, the mixed cells prior to incubation comprise matrix and tumour cells in a matrix:tumour cell ratio from 100:1 to 10:1. The ratio of matrix cells may for example range from 25:1 to 15:1, e.g., from 22:1 to 18:1, e.g., from 21:1 to 19:1, e.g., 95% matrix cells to 5% tumour cells.

In the methods of the invention, the mixed cells may be compacted and/or concentrated (e.g., in step (c) of the method) by any suitable means, e.g., by capillary action, evaporation, centrifugation, gravitation, suction, or aspiration.

In some embodiments, step (c) comprises centrifugation of the mixed cells and removal of supernatant liquid suspension medium. In some embodiments, the mixed cells may be placed upon the support of step (d) after centrifugation and removal of supernatant liquid suspension medium in step (c). The mixed cells may also be placed upon the support of step (d) by centrifugation. Said centrifugation may be the same centrifugation employed in step (c).

In preferred embodiments, step (d) comprises allowing the cells to further compact upon the support. Further compaction upon the support may for example be by evaporation and/or capillary action. For example, the capillary action of step (d) may further compact the mixed cells. The capillary action of step (d) may also compact/and or concentrate the mixed cells in step (c).

That is, compaction of the mixed cells may be achieved by the capillary force exerted by the liquid medium held by capillary action on the second surface (e.g. the contralateral side of the support). This capillary action may cause a reduction in the volume of liquid suspension medium in which the mixed cells of the method of the invention are suspended, and thus of the liquid volume of the organotypic co-culture, thus causing the cells to come into closer contact.

Where the cells are concentrated and/or compacted by suction or aspiration, the mixed cell suspension comprising tumour cells and non-tumour cells may be placed on a first side (surface) of a support and suction applied to a second (e.g. opposite) side (surface) of the support. This may lead to concentration and/or compaction of the mixed cells. The support on which the cells are placed must be adapted to allow the suction applied to one side of the support to be effective at compacting the cells on the other side of the support.

The mixed cells may also be compacted by the action of a pump which applies a hydrodynamic force upon the cells by driving fluid flow through the support. The pump would then be placed on the same side of the support as the mixed cells which are to be compacted.

A method of preparing an organotypic culture from a single cell type or single organ is described in WO 2006/136953, the contents of which are incorporated herein by reference.

As the skilled person would appreciate, incubation of the co-culture of the invention may be done under standard conditions of temperature, humidity and CO₂ appropriate for the cell type. For example, the incubation may be carried out at 20-42° C., e.g., at 22-41, 26-40, 30-40, 32-39, 34-39, or 36-38° C., or at 30-37° C., or 32-37 or 34-37° C. Preferably, the incubation is carried out at 34-38° C., e.g. at 37° C.

Methods of the invention may also comprise a step of obtaining cells from a source. Said source may be a tissue or organ sample. Said source may also be a subject, e.g., a human and/or non-human subject, e.g., a human and/or non-human mammal. The desired tissue or organ sample may be obtained e.g. from a sacrificed mammal, from biopsy material during surgery or from cadaveric material.

The methods of the invention may further comprise a step of cryopreserving the organotypic co-culture. Cryopreservation allows accumulation and storage of cultures to be used for screening purposes. Typically the cryo-preservation is accomplished by freezing at the temperature of liquid nitrogen.

The invention provides in vitro three-dimensional, organotypic cell co-cultures obtainable by the methods described herein.

### Media

Conveniently, the media used according to the invention may be in a liquid form, e.g., a liquid suspension medium or a liquid culture medium. A culture medium according to the invention preferably comprises a complete growth medium that supports the growth of all the cell types being co-cultured. The most suitable medium may vary depending on the organ (or organs) from which the cells in the culture are derived. Preferably the medium provides the nutrients necessary for organotypic growth. Examples of suitable liquid media for brain tissue are described, for example, in Stoppini L. et al (1991) and Muller et al (2001). Other suitable media for other organs are published or can be derived from the culture of cells derived from those organs. The suitable culture medium to use is within the knowledge of a person skilled in the art of tissue culture. The growth conditions and media used are usually those preferred by the matrix cells as the tumour cells are less affected by media composition.

Semi-Permeable Supports and Uses Thereof in Accordance with the Invention

Any suitable support may be used to grow the mixed cell co-culture, provided the support is semi-permeable. The support is thus porous, or semi-porous. The support may be a semi-permeable membrane.

Preferably, the semi-permeable support comprises hydrophilic PTFE (polytetrafluoroethylene; DuPont trade name Teflon®), PET (polyethylene terephthalate) or aluminium oxide (e.g. Anopore™, Whatman Corp.). The support may also consist of hydrophilic PTFE, PET or aluminium oxide. The support may in some embodiments be optically transparent. This allows the use of microscopy with an objective lens to view the cells from either side of the surface.

The support may be coated with a material that facilitates adhesion. The coating may be selected from collagen, laminin, fibronectin or matrigel. Preferably, the support does not consist of a biomolecular, cellular or acellular scaffold, and apart from an adhesive coating does not comprise extracellular scaffold protein or extracellular matrix protein. In preferred embodiments of the invention, the support does not comprise a basement membrane. The support preferably does not comprise a natural or artificial basement membrane. The support preferably does not comprise any scaffold comprising or derived from basement membrane.

According to the methods of the invention, the co-culture may be disposed upon a first surface of the support facing a gaseous phase, and a second surface of the support faces and is in contact with the liquid culture medium. The liquid culture medium is retained in contact with the support and with the culture by virtue of surface tension and/or capillarity. The support therefore preferably has sufficient porosity for liquid media to permeate the support and reach the co-culture on the opposite surface. The liquid culture medium is preferably retained in contact with the support and with the culture by virtue of surface tension and/or capillarity. The co-culture on the support it is not immersed in the liquid culture medium, but is rather covered only by a thin film of the culture medium. This allows better gas exchange between the medium and the co-culture.

### Genetic Engineering

The matrix cells and/or the tumour cells may be genetically engineered. For example, the matrix cells and/or the tumour cells may be derived from a transgenic or otherwise genetically engineered animal (e.g., a non-human mammal, non-human primate, a rodent or a mouse). A transgenic or otherwise genetically engineered animal has a recombinant genome. It has had deliberate modifications made to the genome, rather than those that occur naturally.

Generally, either tumour or matrix cells of the co-culture may be genetically engineered. That is, they may be recombinant. Herein, the terms “genetically engineered” and “recombinant” may be used interchangeably.

In certain preferred embodiments, the tumour cells are genetically engineered. The tumour cells may have been transformed by genetic engineering, i.e. cancerous properties may be conferred upon the tumour cells by genetic engineering. Alternatively or additionally, the tumour cells may be engineered in a different respect, such that they contain a genetic alteration in addition to the genetic features which confer cancerous properties upon the tumour cells. For example the tumour cells may be genetically engineered to carry a reporter gene or resistance gene. In certain preferred embodiments, the tumour cells are thus labelled with a reporter system.

The matrix and/or tumour cells can be genetically engineered prior to setting up the organotypic culture. The genetic engineering can for example be performed upon dissociated suspensions of these cells. Generally, one or more transgenes can be introduced by means of transfection or transduction in an appropriate vector. A vector expressing siRNA may also be introduced.

Genetic engineering of the cells within the co-culture can be applied to many aspects of the models applications, for example, the cells may be genetically altered to modulate expression of a drug target or a biomarker. A biomarker is a molecular marker, the presence of which at a certain level or in a certain molecular form indicates the presence of a diseased state. A drug target is a molecular species that can be modulated to affect a disease process, i.e. a molecule through which a drug acts. In drug development changing the nature or level of function of the drug target must have a positive impact on disease outcome, and the target should be of a molecular type that is amenable to modulation. In many cases, information about drug targets is obtained from genetic and other biological studies, and classes of compounds that are known to interact with those targets are available. It is often desirable to modulate the levels of these biomarkers and drug targets in biological systems, and to study the biological consequences. Sometimes combinations of drugs can be used which act on the same target or can act on several different targets in multiple cell types and/or neighbouring cell types

The tumour cells may also be genetically altered to express a visual marker, such as a fluorescent marker, that allows the cells to be tracked visually. This allows the tumour cells to be instantly identified during live imaging.

Technologies to express cloned genes and to ablate the expression of cloned or endogenous genes are known in the art. These technologies may be used to increase or decrease expression of a marker, such as a drug target or biomarker, in the cells used in the methods of the invention. For example, the expression of a drug target may be modulated in selected dissociated cells before the cells are compacted and the organotypic culture is prepared. This approach is much more efficient than attempting to alter expression of a drug target in the final organotypic culture because single dissociated cells can be manipulated much more easily.

Techniques to increase expression of a cloned or endogenous gene are based on the introduction of heterologous DNA in a form which recruits the cellular expression system, and many different approaches are well known to those skilled in the art. In some cases naked DNA may be used with a lipophilic transfection reagent, the DNA including a strong promoter co-linear with the gene to be expressed and a replication origin that enables cytoplasmic replication of the introduced DNA. In other cases a viral vector may be used to increase the efficiency of DNA introduction. Similarly, means to ablate gene expressions that are well known to those skilled in the art including antisense DNA oligonucleotides, peptide nucleic acid and double-stranded RNA interference. In some cases, naked nucleic acid may be used. In other cases, especially for the use of small interfering RNA, expression vectors may be used to express the molecule in a self-assembling hairpin form. It has also been shown that proteins can be introduced directly into cells provided that they are attached to an entity that encourages transport from the exterior to the interior of the cell. The Tat protein of human immunodeficiency virus (HIV) is one such entity, and proteins to be transferred may be produced as fusion proteins with HIV-Tat and introduced into cells (Becker-Hapak M. et al, 2001).

It will also be clear to those skilled in the art that, instead of transforming or transfecting the matrix cells as described above, the organ derived cells used in the method of the invention may be from a transgenic animal. For example, the organ derived cells may be from a transgenic animal expressing a visual marker, such as a fluorescent marker, of from a transgenic animal in which expression of a particular drug target or biomarker has been increased or decreased.

In certain preferred embodiments of the co-cultures and methods of the invention, the tumour cells are not genetically engineered. For example the tumour cells may be derived from primary tumour cells or from cancer cell lines which have become transformed without a genetic engineering step. In some embodiments, the matrix cells are not genetically engineered. In some embodiments, neither the tumour cells nor the matrix cells are genetically engineered.

### Applications, Uses, Kits and Devices of the Invention

The co-cultures of the invention may also be used in methods for screening for anti-cancer agents. Thus the invention provides a method comprising contacting a co-culture of the invention with a test agent, wherein an inhibitory effect of the test agent upon the growth, proliferation, viability, survival, or migration of the tumour cells of the co-culture indicates that the test agent is a candidate anti-cancer agent.

The invention also provides the use of a co-culture of the invention in a method of identifying and/or validating a cancer biomarker and/or a cancer drug target.

Such screening, identification or validation methods may also comprise the preparation of co-cultures according to the methods described herein.

The co-cultures and methods described herein may be used in high throughput formats, e.g., for screening (or secondary screening) of anti-cancer compounds.

In a further aspect of the invention there is provided an assay kit comprising one or more co-cultures according to the invention.

In another aspect of the invention there is provided a device for screening compounds comprising one or more co-cultures according to the invention.

A suitable device that can be adapted for use in the methods described herein is described in WO 2006/134432, the contents of which are incorporated herein by reference in their entirety. The device may contain one or multiple cultures according to the invention. The device may have a medium conduit having one open end and one end closed by a semi-permeable support (e.g., a porous membrane) on which the co-cultures can be grown, according to the methods of preparing the co-cultures described herein. The co-culture of the present invention may be placed upon a first surface of the support, the medium conduit being situated on a second surface of the support, such that the co-culture may face a gaseous phase, as described herein. The medium conduit may be supplied with (or may contain) the liquid culture medium, in order to supply nutrients to the co-culture. The liquid culture medium may be retained in the medium conduit, and in contact with the support and the co-culture, by surface tension and/or capillarity.

Each culture may be maintained on a separate surface or on a separate section of a large surface and nourished separately. The device may be adapted to accommodate multiple parallel co-cultures per device, preferably 2, 4, 8, 16, 24, 96, 384, 1536 or more parallel cultures per device, or multiple devices, preferably 2, 4, 8, 16, 24, 96, 384, 1536 devices, each carrying an individual co-culture, may be used in parallel.

One or more co-cultures can also be incorporated in assay kits for testing a variety of factors in the study of cancer such as testing the efficacy of prospective anti-cancer compounds.

A co-culture, a device or an assay kit of the invention may further comprise means for monitoring physiological or environmental parameters in the co-cultures of the invention, such as electrophysiological parameters or cell metabolism, and in particular any indicator of cell viability or survival. Such means allow the cellular environment to be monitored, e.g. during the growth or maintenance of the co-cultures, or during screening, identification or validation methods described herein, e.g., during screening or testing of candidate anti-cancer agents.

Means for monitoring physiological or environmental parameters in the co-cultures of the invention comprise means for measuring electrophysiological parameters or response (for example electrodes) and biosensors.

A biosensor may monitor cellular metabolism and the extracellular environment within the co-cultures. For example, one or more biosensors may monitor one or more metabolic parameters and/or metabolites in the co-cultures of the invention, metabolic parameters and/or metabolites may be intracellular or extracellular. For example, a biosensor may monitor at least one metabolic parameter or metabolite selected from lactate, glucose, glutamine and pH. A biosensor may also monitor any indicator of cell viability or survival. Indicators of cell viability or survival are commonly known in the art. For example, a biosensor may monitor lactate dehydrogenase (LDH) as an indicator of cell survival.

The co-cultures can thus be used to monitor physiological changes in the environment of tumour cells as they grow in a non-tumour cell matrix, or to monitor effects of manipulating the cellular environment upon the growth, structure, viability or survival of tumour cells. The formation of aggregates and migration of the cells can of course also be monitored. Gas penetration and/or gas exchange may also be monitored, e.g., oxygen tension. As the cultures are thin, it is possible to manipulate the oxygen tension through the culture to study the effect of varying degrees of hypoxia on the cultures. The degree of hypoxia within tumours is known to be an important factor in tumour formation, invasion and growth.

The co-cultures of the present invention are simple to use, maintain and manipulate. The monitoring and measurements described herein may be done in real time. The monitoring and measurements described herein may also be done without invasive procedures. Co-cultures can therefore be monitored over time, e.g., by imaging without being damaged.

The co-culture model of the present invention allows for ease of harvesting material over time. Samples from a single batch of cultures can be harvested for RNA, DNA, and protein and immunohistochemical analysis over a period of weeks. This is an advantage over xenografts where harvesting tissue generally requires sacrificing animals at each time point. Thus, the present invention allows the reduction, refinement and replacement of animal experiments, thereby reducing costs where large studies are required and minimising ethical considerations.

A further application of the co-culture of the present invention is the ability to investigate the pathways involved in cell to cell interaction or drug treatment in greater detail. The inventors have shown herein that the tumours develop by migration of the tumour cells as opposed to the clonal expansion of the cells within the tissue by using red and green expressing cells of the same tumour cell line and mixing them in equal ratio into the same co-culture of brain cells (see Example 5). If the cells were clonally expanding the cells would produce a large number of single coloured aggregates. However it was observed that the aggregates are few in number compared to the initial seeding and that both red and green cells are present in the resultant aggregates. This observation strongly suggests migration of cells within the matrix in response to cell signals from the matrix cells. The pathways involved in these migrations can be analysed to see what effect the inhibition has on the migration. For example, such analysis may be carried out using RNAi or other specific inhibitor compounds to inhibit cellular processes of interest. Commonly known methods of DNA, RNA and protein analysis can be used to show which genes are expressed during the cell and tumour interactions. The co-cultures of the present invention are robust and easy to manipulate and are therefore very amenable for all these methods of analysis.

The response to cell signals will depend on the normal function of the cells. In some cases, the cells that respond to such signals may respond with the extension of cellular processes such as the axonal processes of neurons. In other cases, the cells may respond with cellular processes that lead to cellular movement through the culture either towards or away from the cell or cells producing the signal. Alternatively, the cells that respond may respond with cell division or inhibition of cell division that they might otherwise undergo. The cells that respond may also respond by producing other signals causing secondary responses on cells that were initially unresponsive to the initial signal. In this way, various changes in cell number, function and distribution may occur within the culture during the period of culture, reflecting the organotypic behaviour of the culture.

The co-cultures of the invention may be used to study the way primary tumour cells behave in, and interact with, matrix cells of the tissue from which they derive and how they behave in, and interact with, matrix cells of a tissue to which they metastasise. Metastasis is the process which allows tumours which develop in one tissue to migrate to and invade another tissue type. For example, breast tumours develop in breast tissue but can metastise to other organs, for example brain. Differences in the behaviour and properties of cells, in particular tumour cells, in the co-culture of the present invention provide important insights into the metastasis process.

The co-culture model of the present invention can be used for testing compounds or treatment regimes that may be effective in tumour reduction or tumour cell death. It is possible to monitor tumour reduction after the tumours have formed within the model or to test the efficacy of compounds at reducing tumour cell numbers. These tests can be performed on single compounds or on treatments with panels of compounds where synergistic effects may occur. Large numbers of compounds at different concentrations and in different combinations can be tested quickly. The cell to cell interactions also allow simultaneous study of the toxicity of the compounds on the non-tumour matrix cells to be examined. The interaction of the tumour cells with the matrix cells allows for testing compounds in a model that more clearly represents that found in a cancer patient. This type of assay not only removes compounds that will be ineffective in vivo despite being effective against monocultures of tumour cells in either 2D or 3D but can also indicate compounds that will be effective in vivo despite being ineffective in monoculture. It is also possible to test compounds to see the effect they have on tumour regression by allowing the tumour cells to aggregate before addition of the compounds. This is important in the treatment of tumours which cannot be excised surgically.

Screening of several molecular classes, such as proteins and lipids, in organotypic cultures that express a disease state or the corresponding non-diseased state may be used to identify biomarkers. Validated biomarkers are currently used both to identify carriers of a disease state and to monitor their progress towards normality that may be assisted by a therapeutic regime such as a drug. It is necessary to establish a statistically significant association between a candidate biomarker and a disease state to validate the biomarker for use in clinical trials. The organotypic cultures of the present invention are ideally suited to biomarker discovery and validation due to the fact that they replicate organ function and physiology and can be generated quickly and easily by the methods of the invention such that they are applicable to high throughput assays. The organotypic cultures of the invention could thus be used much more rapidly and cheaply than whole animals currently used for the identification and validation of biomarkers.

The tumour cell co-cultures can be used to identify and/or validate biomarkers and/or drug targets (e.g., cancer biomarkers and/or cancer drug targets). Assays for identifying biomarkers and/or drug targets include the use of transcriptional profiling, proteomics, mass spectrometry, gel electrophoresis, gas chromatography and other methods for molecular profiling known to those skilled in the art. Surrogate markers are a sub-set of biomarkers that can be used to assess the presence or progression of a disease state, but that do not measure directly a clinical outcome of the disease. Where these surrogate markers show a response that correlates with drug treatment they are referred to as pharmacodynamic markers. The organotypic tumour co-cultures of the invention may be used to identify and validate these pharmacodynamic markers in the same way as other biomarkers, e.g. in the study of cancer.

Embodiments of the present invention will now be described by way of illustration only in the following examples in which:

### BRIEF DESCRIPTION OF THE DRAWINGS

FIG. 1: FIG. 1A is a schematic diagram of the method for producing a co-culture according to the invention. A disaggregated suspension of matrix cells is prepared and mixed with tumour cells, e.g. by adding tumour cells to the matrix cells. The mixed cells are placed on a support at the gas liquid interface enabling the cells to be compacted by capillary action. FIG. 1B shows a transverse section of an organotypic culture prepared from dissociated brain cells 20 days after seeding. Beta-Tubulin (green) and GFAP (red) antibodies were used to detect neurons and astrocytes respectively. Nuclei are labeled blue by DAPI (4′,6-diamidino-2-phenylindole) staining. The brain cell cuture are positive for choline acetyltransferase (ChAT; FIG. 1C), astrocytes (GFAP; FIG. 1D) and neurons (beta-tubulin; FIG. 1E). FIG. 1F shows the merged images of the GFAP and beta-tubulin detection. FIG. 1G shows that spontaneous and evoked electrical activity can be observed in the co-cultures with optimal activity at 14 DIV (Days in vitro).

FIG. 2 contains micrographs showing the growth of human tumour cells from cell line LN-18 in the rat brain cell matrix at day in vitro (DIV) 0 and DIV 4 after preparation at different concentrations of tumour cells (1%, 5% and 10%) in the rat brain cells matrix. The tumour cells, which express GFP (green fluorescent protein), appear white in the micrographs and by DIV 4 at 10% tumour cells to 90% rat brain cells it can be seen that aggregates or clumps of tumour cells have formed. Less clumping is seen at the lower concentrations.

FIG. 3: FIG. 3A contains micrographs showing the growth of LN-18 tumour cells in the rat brain cell matrix at DIV 1, DIV3 and DIV 14 after preparation of the co-culture. The tumour cells express GFP and appear white in the brain cell matrix. The tumour cells show some aggregation at DIV 3 and remain aggregated at DIV 14. Figures B, C, D: eGFP-labeled GL-15 (B), A172 (C), U87 (D) cells were allowed to grow in co-cultures with brain cell matrices according to the invention.

FIG. 4a is a micrograph showing the growth of SKOV 3 human ovarian tumour cells in the rat brain cell matrix at day 6 after preparation of the co-culture. The SKOV3 cells express GFP and appear white in the brain cell matrix. The cells are concentrated at the periphery of the organotypic co-cultures.

FIG. 4b shows the proliferation of SKOV3 cells at day 1, 3 and 7 and in different matrix cells. The cells expand at a greater rate in a matrix of fibroblast and endothelial cells (white blocks) than they do in the rat brain matrix (black blocks).

FIG. 5 is a micrograph showing the growth of tumour stem cells in rat brain cell matrix at day 1 and day 2 after preparation. The cells appear to have differentiated and formed processes within the matrix.

FIG. 6 contains graphs showing the differences in efficacy of temozolomide (TMZ) on 2D mono-layer cultures of tumour cells alone compared to the 3D organotypic tumour co-culture prepared according to the present invention. The results show that there was little response in terms of reduction of tumour cell numbers to the compound at levels up to 10 μM in the 2D culture (FIG. 6a) compared to the untreated control cells, whilst in the 3D model the IC₅₀ (the concentration of TMZ at which the cell number is halved) appears to be less than 5 μM (FIG. 6b).

FIG. 7 contains graphs showing the differences in efficacy of paclitaxel (Taxol) on 2D mono-layer cultures of tumour cells alone compared to the 3D organotypic tumour co-culture prepared according to the present invention. It can be seen that a reduction in cell number to almost zero is achieved with concentrations of Taxol of 20 nM in the 2D culture (FIG. 7a) whilst in the 3D model the IC₅₀ (the concentration of Taxol at which the cell number is halved) appears to be approximately 25 nM (FIG. 7b).

FIG. 8 contains graphs showing the differences in efficacy of Cytosine arabinoside (Ara-C) on 2D mono-layer cultures of tumour cells alone (FIG. 8a) compared to the 3D organotypic tumour co-culture prepared according to the present invention (FIG. 8b).

FIG. 9 compares the effects of Taxol and Temozolomide when applied to tumour co-cultures of the invention either at seeding (A and B) or 3 days after cell seeding (C and D), when the co-cultures and tumour cell aggregates are established.

### EXAMPLES

### Example 1

### Preparation of Human Tumour Cells

Human glioblastoma tumour cell lines, LN-18 (ATCC #CRL2610) were cultured and were transfected with either green fluorescent protein (GFP) expressing plasmids or red fluorescent protein (RFP) expressing plasmids. Cells were cultured and plated onto 6 well plates, at 1×105 cells per well, grown overnight and checked to see ˜75% confluence. The cells were transfected with pEGFP-N3 (BD Biosciences cat no #6080-1) using Gene Juice (Novagen cat no #70967-EA) as per manufacturer's protocols. 24 hours after transfection, the cells were selected with Geneticin (GIBCO cat no #10131-019), a population of selected cells were used in the experiments. A population of selected cells were cultured over prolonged periods of time with occasional selection to maintain a population of transfected cells. The expression of fluorescent proteins by the tumour cells allows monitoring of the tumour cells for proliferation and morphological changes. The transfected tumour cells (LN-18) were treated with trypsin/EDTA (sigma#T4299) to remove them from the flask, resuspended in culture media, The viable cells were counted using trypan blue exclusion The suspension was centrifuged at 1000 rpm/3 minutes and the resultant cell pellet resuspended to 50,000 cells/μl.

### Example 2

### Preparation of Rat Brain Cells from Isolated Rat Cortex

The organotypic cultures were produced by preparing dissociated cells from isolated rat cortex. P0 Wistar rats were sacrificed and the brains removed. The cortices were dissected into a Petri dish and chopped into small pieces and put into cold EBSS (GIBCO cat no #24010). The tissue pieces were then triturated very gently. The resultant cell suspension was filtered through a 100 μm mesh filter fitting into a 50 ml falcon tube. The suspension was centrifuged at 1000 rpm/3 minutes. The viable cells were counted using Trypan blue exclusion and the cell pellet resuspended to 50,000 cells/μl.

### Example 3

### Establishment of 3D Organotypic Cultures from Dissociated Brain Cells

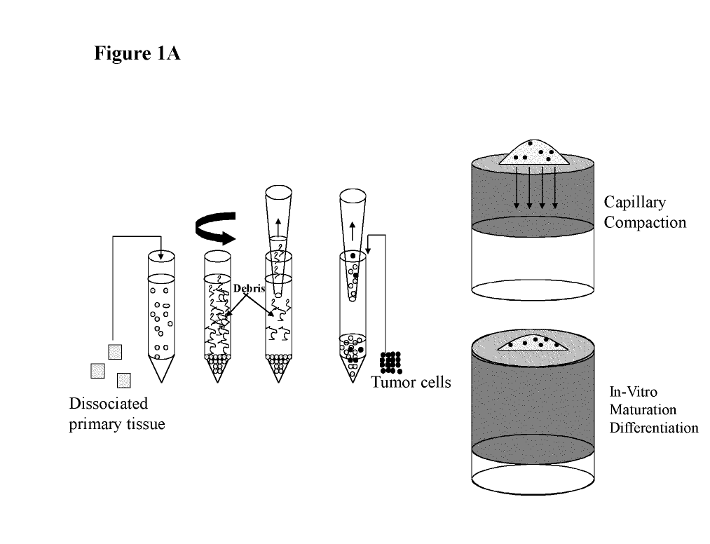

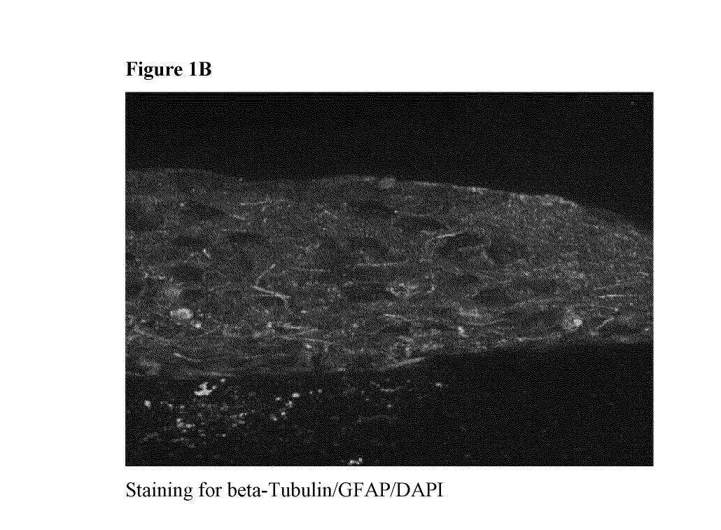

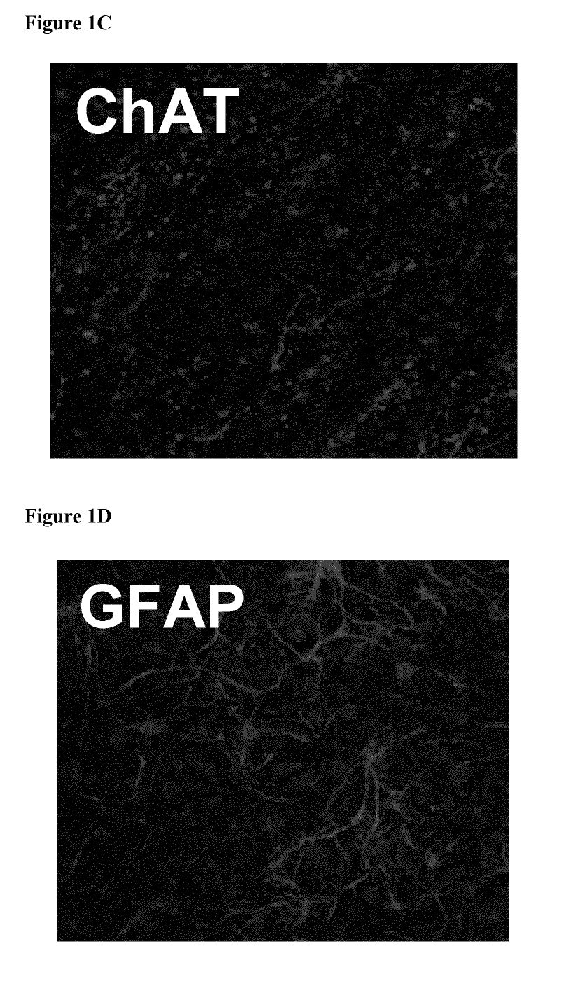

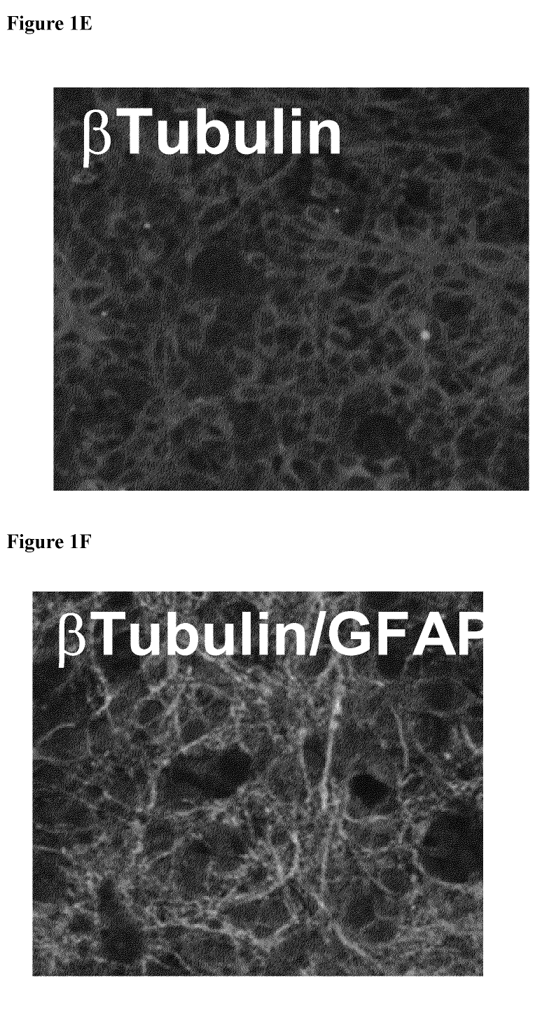

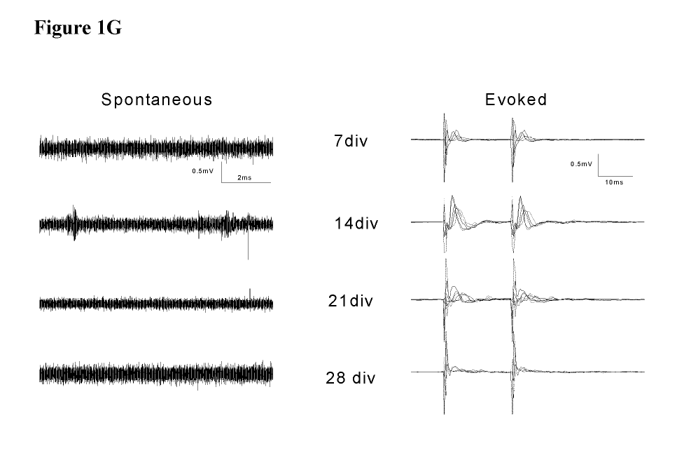

Dissociated rat brain cortices were allowed to re-aggregate on a semi-porous membrane at an air-liquid interface (cf. FIG. 1, right hand portion). Freshly dissociated brain cells from rat embryo were plated on hydrophilic PFTE membranes placed on media and allow to grow for 20 days. A transverse section of the cell masses obtained was processed for immunofluorescence using markers for neurons (beta-tubulin) and activated astrocytes (glial fibrillary acid protein, GFAP). As shown in FIG. 1B, the staining revealed that both neurons and astrocytes were present in such 3D cultures and that their physiological architecture was preserved (see also FIGS. 1 D, E, F). Cholinergic neurons and nerve terminals were subsequently visualized using an antibody directed against choline acetyltransferase (ChAT; FIG. 1C). As expected, ChAT staining was distributed throughout the whole brain co-culture with specific enrichment in the nerve terminals. The presence of mature neurons establishing connection was further investigated by measurement of electrophysiological parameters. Up to four weeks after brain co-culture seeding, spontaneous and evoked electrical activity could be observed with an optimal activity fourteen days after start of the experiment (FIG. 1G). The propagation of such electrical signals are typical of a group of interconnected neurons.

### Example 4

### Preparation of a 3D Organotypic Co-Culture from Dissociated Matrix and and Tumour Cells

The transfected tumour cells and the rat primary cells prepared as explained in Examples 1 and 2 were mixed together to form a suspension of cells at 50,000 cells/μl.

5 μL of this mixed cell suspension was added to the top of a 0.4 micron Millipore culture insert (Millipore cat no # PICM0350) placed in lml cortical media (10% Hams F12, 20% FCS, 5% horse serum, 10 mM Hepes, 2 mM glutamine in DMEM) in a 6 well plate (NUNC). The plate was then incubated at 37° C. with 5% CO₂ (FIG. 1A). During the first hours of this incubation the cells compact to form a denser culture by the removal of the liquid from the 5 μl cell suspension through capillary action via the semi-permeable membrane support (FIG. 1A).

### Example 5

### Optimisation of the Concentrations and Proportions of Matrix and Tumour Cells in the Organotypic Co-Culture

Optimisation is desirable as at too low concentration the cells may fail to form tumour-like aggregates and at too high concentrations the cells may form aggregates too rapidly and the process of cell migration cannot be observed. When the cells are mixed at appropriate concentrations and proportions the tumours form over 3-4 days and do not cause the destruction of the rat brain matrix.

The rat brain cells were optimised for cell number to allow optimal oxygen penetration through the tissue and formation of a compact organotypic structure after capillary compaction. The cells were resuspended at concentrations of 25, 50, 75 and 100 thousand cells per μl and 5 μl of these suspensions were plated as explained in Example 4. The resultant cultures were monitored microscopically for signs of hypoxic damage indicated by dark areas within the culture and compact organotypic appearance. The optimal cell number for these cultures was 50,000 cells/μl with 5 μl used for each culture. It should be noted that preparing the organotypic co-cultures using matrix tissue other than rat brain would require optimisation in this manner for cell numbers as required.

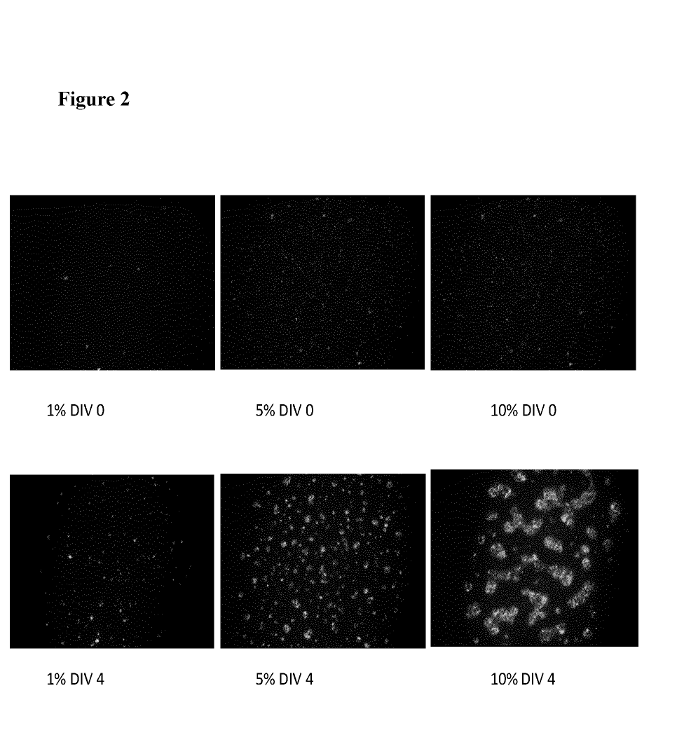

The LN-18 tumour cells were tested for the ability to form tumours at concentrations of 1, 5 and 10% (% of the total number of cells). At 1% the cells do not seem to migrate into tumour-like clumps (probably due to a weak gradient of chemokine concentration). At 5% concentration the cells form clumps at about 3-4 days. At 10% concentration the clumping is more rapid and the clumps are more compact and cause some disruption to the rat brain matrix (FIG. 2). The clumping does vary with cell line. Some cell lines clump very rapidly and strongly, some clump more slowly and the clumps will tend to disperse over time. It is therefore necessary to optimise the ratio of tumour and rat cells for each cell line used.

Using these optimisation experiments the cells were mixed in the proportions of 95% rat brain cells and 5% LN-18 tumour cells both resuspended at 50,000 cells per μl.

### Example 6

### Morphology of the Co-Culture of the Rat Brain and the Tumour Cells in the Organotypic Co-Culture

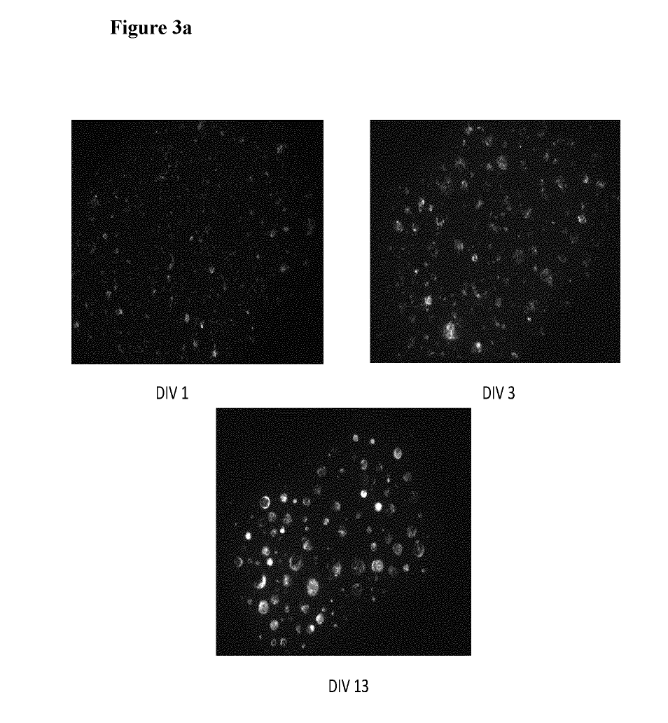

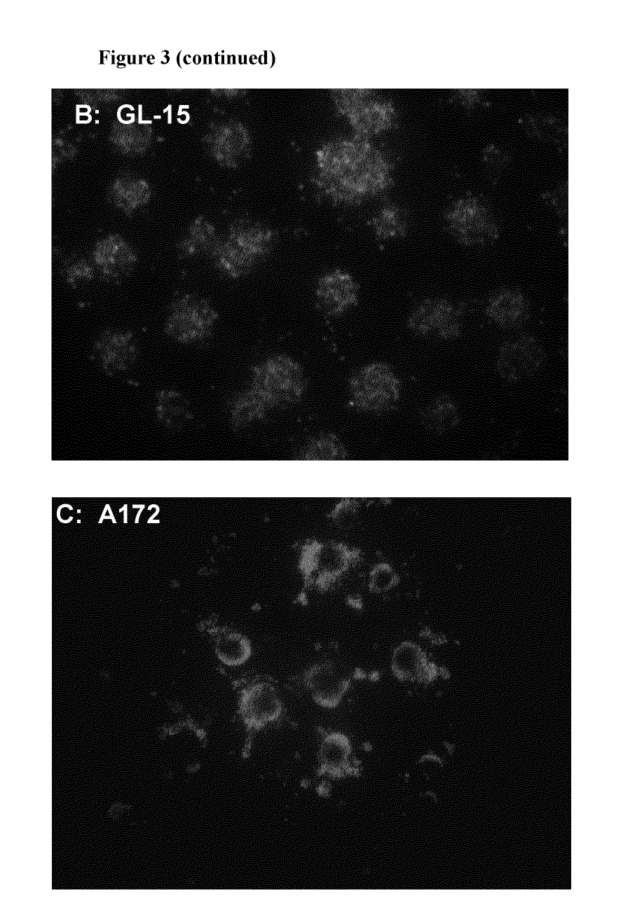

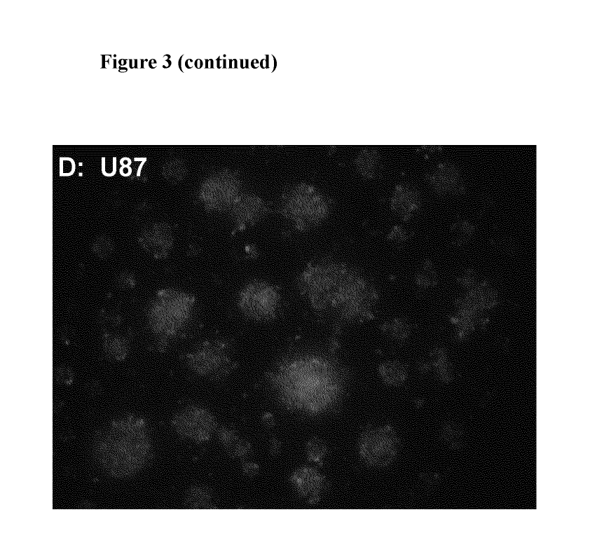

In one experiment, the growth of the LN-18 tumour cells within the organotypic co-cultures using the optimal concentration 5% to 95% tumour to rat cells was monitored microscopically over a period of 13 days (FIG. 3). By day 3 in vitro (DIV) the cells had formed tumour-like aggregates in the primary rat tissue. These tumour-like aggregates were still present after 13 days in vitro (DIV).

In a further experiment, LN18 cells stably transfected with a vector encoding the eGFP protein were allowed to grow within rat brain matrix cells and were observed beyond day 13. One day after seeding, individual LN18-eGFP cells could be observed spread throughout the co-culture. Three days later, the number of eGFP positive foci decreased while the size of the foci (“cell aggregates”) increased. At day 7, the number of eGFP positive foci only slightly diminished while foci still expanded in size. The foci size reached a maximum at day 8. Fourteen days after incorporation the cells were still aggregated. The aggregates have been observed to be present at least until day 25.

### Example 7

### Formation of Tumour Co-Cultures with Further Glioblastoma Cell Lines

Similar effects were seen with glioblastoma cell lines GL15, A172 (ATCC#CRL1620) and U87 (ATCC#HTB-14). Each of these cell lines was eGFP-labeled and mixed with rat brain cells to form 3D organotypic co-cultures. As indicated by the eGFP signal, all brain-derived tumour cell lines grew and formed clusters of tumour cells within the co-cultures (FIGS. 3B, C and D) in a very similar way as LN18 cells.

### Example 8

### Characterisation of Tumour Cell Migration and Aggregation in Co-Cultures of Rat Brain and LN-18 Tumour Cells

Experiments were performed to see if the tumour-like clumps in the organotypic co-cultures developed from clonal expansion of the cells or from the migration of cells through the cell matrix. Human brain tumour cell line LN-18 was transfected to produce cells either eGFP or RFP. LN18 RFP expressing cells and LN18 eGFP expressing cells were mixed together and then mixed as a 5% LN-18 to 95% rat brain cell ratio with dissociated rat cortex cells and plated onto the porous membrane to form a compact 3D organotypic co-culture as previously described. These cultures were monitored over 14 days. 24 hours after being incorporated into the co-cultures, the cells appear as a homogeneous mixture of green and red cells. As early as 48 hours after incorporation some cell clusters started to be formed. Four days after incorporation, cells clearly formed aggregates of mixed red and green cells. The fact that the red and green cells co localize demonstrate an active clustering of tumour cells together rather than a clonal expansion of single tumour cells. Thus the cells appeared to migrate to form the tumour-like aggregates. If the effect was due to clonal expansion of the cells it would be expected that individual red and green tumours developed rather than the mixed red and green tumours that were observed. This is evidence that the cell matrix provides an environment that allows interactions of and with the tumour cells in a way that more closely represents an in vivo situation.

### Example 9

### Morphology of the Co-Culture of the Rat Brain Cells and Other Tumour Types in the Organotypic Co-Culture

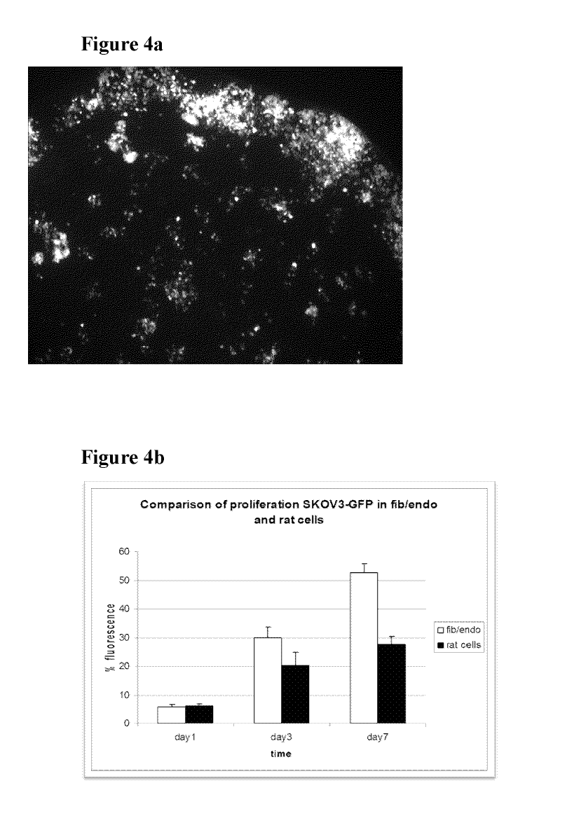

Human ovarian cancer cells, SKOV3 (ATCC# HTB77) did not form tumours in rat brain cell matrix (FIG. 4a). This was tested by mixing transfected SKOV3-GFP cells with rat brain cells as described for the glioblastoma cells in Example 4. The SKOV3 cells also do not aggregate in simulated ovarian tissue, however, the cells do grow in both cell matrices.

Endothelial cells (ATCC# CRL 2299) and human fibroblasts (Promocell# HPF-c #12360) were grown according to standard operating protocols set out by the suppliers, and the cells were removed from the flasks with Trypsin (Sigma#T4299) and then neutralized with trypsin inhibitor (Sigma#T6414). The cell suspension was centrifuged at 1000 rpm/3 minutes. The viable cells were counted using Trypan blue exclusion and the cell pellet resuspended to 50,000 cells/μl. This cell suspension was used to replace the rat brain tissue in production of organotypic co-cultures as described in Example 4. These cultures were also grown on confetti (small circles of membrane which sit on the supporting membrane and allow ease of handling) that had been treated with laminin as this aided the adhesion of the cells. These cells tend to aggregate at the periphery of the primary non-tumour cell matrix. However the system still allows analysis of the growth of these cells (FIG. 4b) and detailed analysis of metabolic pathways. This indicates that the model can be applied to tumour types other than Glioblastoma tumour types even when solid tumours are not formed. Analysis of pathways and drug susceptibility is still possible.

### Example 10

### Organotypic Co-Cultures Formed from Rat Brain Cells and Tumour Stem Cells

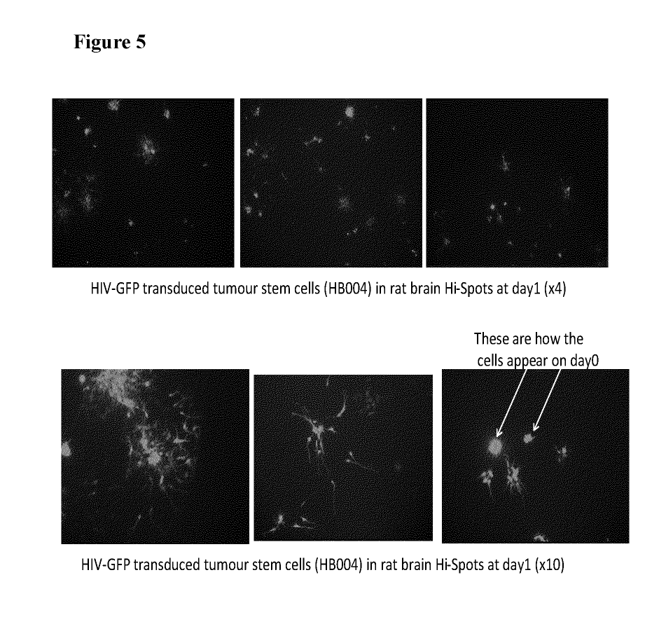

Tumour stem cells were isolated from biopsy material (a kind gift from W. Gray) the cells were cultured in flasks and then the cells were transduced with the lentiviral vector rHIV PPT-PGK-GFP-WPRE (VSVg) (Genethon). 10 μl of vector (˜20×10⁶ infectious particles) was added to 50 μl of suspended stem cells (˜10×10⁶ cells) to give a MOI of 2:1 the cells were then cultured for 1 week and monitored for GFP expression. These transduced tumour stem cells were used in place of tumour cell lines to produce organotypic co-cultures as described in Example 4. During culture in the organotypic co-cultures the cells differentiated and sent out processes (FIG. 5).

### Example 11

### Immunohistological Examination of the Organotypic Tumour Co-Culture

The organotypic co-cultures are easily prepared for immunohistological analysis which allows for complex studies of the interaction of matrix and tumour cells and of the metabolic pathways that may be involved. The organotypic co-cultures were collected and fixed in 4% Paraformaldehyde (Sigma#P6148) then washed 3×5 min in Tris buffered saline (TBS) with 0.001%). —Tween (Sigma#P5927) (TBS_T). The non specific binding sites were blocked by incubation for 30 minutes in 5% normal donkey serum followed by 3×5 min washes in TBS-T. The primary antibody GFAP (Sigma#G3893) 1:200 produced in mouse was diluted in TBS-T, added to the organotypic co-cultures and incubated overnight at 4° C. After 3×5 min washes in TBS-T the secondary antibody Alexafluor goat anti-mouse 568 (red) (Invitrogen # A11031) @ 1 in 500 was added for 1 hour at room temperature in the dark. The organotypic co-cultures were washed 3×5 min in TBS-T then placed on glass microscope slides with mounting fluid (Sigma#F4680). A cover slip was placed on top and sealed with nail varnish. The slides were kept in the dark at 4° C. until imaged. The prepared slides were imaged on an Olympus1X70 microscope with a Becton Dickenson CARV confocal cell imaging system attached.

### Example 12

### Characterisation of Activated Astrocytes in Co-Cultures of the Invention

A distinctive feature of gliablastoma multiforme (GBM) is the presence of reactive astrogliosis surrounding brain tumours. To determine whether the co-cultures of the invention prepared as described in Example 4 could recapitulate this feature of human tumours, expression of the glial fibrillary acid protein (GFAP), a marker of activated astrocytes, was assessed in the GBM/brain co-cultures using a GFAP antibody.

Increased GFAP staining could be observed specifically around and within the tumour cell mass suggesting that astrocytes in close contact to tumour cells are activated. This association of the tumours with GFAP positive cells is important for the production of physiologically relevant tumour models, as the action of some compounds can be affected by the protective effect of these bystander cells.

### Example 13

### 5-ALA Staining of Co-Cultures of the Invention

5-aminolevulinic acid (5-ALA) can be used to visualize tumourous tissue during neurosurgical procedures. The intraoperative use of this guiding method may reduce the tumour residual volume and prolong progression-free survival in patients suffering from brain tumours. GBM(LN18-eGFP)/brain co-cultures as prepared in Example 4 were stained with 5-ALA to see whether, as in human tumours, 5-ALA could specifically stain the tumour cells.

Aminolevulinic acid hydrochloride (ALA) (Sigma product number A3785) was solubilised in water to 10 mg/ml. For incubation with the cultures, it was diluted in the medium in the well to give a final concentration of 100 μg/ml and incubated with the cultures for 24 hours. The cultures were then examined microscopically using the CARV confocal system described for immunohistological analysis.

As shown FIG. 4A, a strong specific 5-ALA staining could be observed in LN18-eGFP clusters of more than about 100 μm in diameter. The tumours appeared green due to the expression of GFP by the tumour cells and the 5-ALA localised with these cells and appeared red when imaged. Tumour cell clusters smaller than about 100 μm in diameter were stained more weakly and sparse cells remained negative.

The 5-ALA staining characteristics indicates a similar reaction to that which be seen in vivo. They provide further evidence that the co-culturesof the invention recapitulate significant properties of in vivo tumours and thus further underline the advantages of this system over the culture of two-dimensional cell monolayers.

### Example 14

### Measurement of pH within the Organotypic Tumour Co-Cultures

Malignant glioma margin tissue are metabolically extremely active, producing high levels of lactate, resulting in acidification of the extracellular space of normal tissue around the tumour edge. To check whether this trait is reproduced by tumour co-cultures of the invention, organotypic GBM/brain co-cultures were prepared as described above (e.g. in Example 4) but using untransfected LN18 tumour cells that did not express eGFP, so as not to interfere with the pH dye staining. The cultures were incubated for 3 days. Then the inserts were removed from the culture media and placed into wells containing sterile Hanks buffered salt solution (HBSS). After an initial wash of 15 minutes, the inserts were removed to wells containing HBSS and 1 μl of 10 mM BCECF acid (2′,7′-bis-(2-carboxyethyl)-5-(and-6)-carboxyfluorescein, mixed isomers), a dye whose fluorescence emission is pH sensitive. When excited at 514 nm, its emission maximum is at 630 nm in a basic environment (green staining) and at 570 nm when acidic (red staining), i.e. more acidic conditions in the cells appear red whilst the neutral pH appear green. The cultures were incubated for 30 minutes and then washed twice in HBSS prior to imaging in the red (emission wavelength 610) and green spectra (emission wavelength 525).

A strong acidic pH (red staining) could be detected around the non-labeled tumour cells while the totality of the brain co-culture was basic (green staining). In the absence of LN18 cells, no acidic pH could be detected in the brain co-cultures. Thus, LN18 cells acidify their close environment when grown in brain co-cultures. These data again show that co-cultures of the invention recapitulate important features of tumours in vivo. These pH changes have important implications for the activation of some anti-tumour compounds and could be critical in determining clinical efficacy of some test compounds.

### Example 15

### Measurement of Response of Tumour Cells to TMZ. Comparison of the 3D Organotypic Tumour Co-Culture with 2D Monolayer Cultures of Tumour Cells

The organotypic co-cultures were used to analyse the response of the tumour cells to anti-tumour compounds. The standard of care for glioblastoma includes Temozolomide (TMZ) chemotherapy during and after radiotherapy. Here the inventors have tested the efficacy of TMZ (Sigma#T2577) treatment in organotypic co-cultures compared to standard 2D cultures of LN-18-GFP cells.

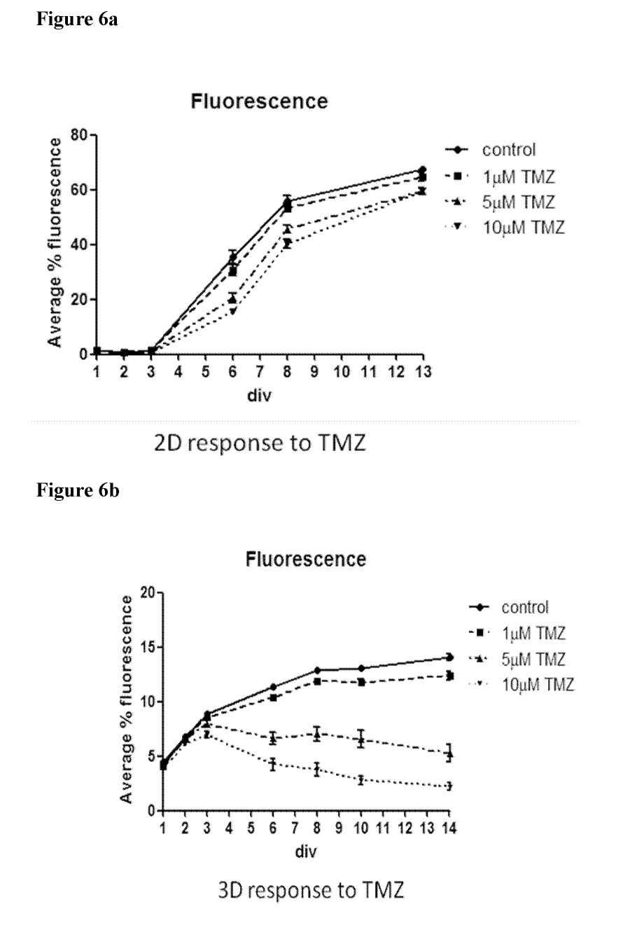

Organotypic co-cultures were prepared as previously described. 2 hours after preparation, TMZ was added into the culture medium at various concentrations—see FIG. 6. The cultures are incubated in the culture medium+TMZ for up to 14 days with the compound being renewed as the media was changed. This allowed for monitoring of the effect of the TMZ over time.

For the monolayer cultures of tumour cells alone (2D) LN-18_GFP cells were plated into 24 well plates at a concentration of 10⁴ cells per well and after 2 hours treated with the same concentrations of TMZ as the organotypic co-cultures. Control organotypic co-cultures and 2D cultures were also monitored to compare normal growth of the tumour cells with that after addition of the TMZ. For analysis of fluorescence, images were taken on a Leica DM1L microscope using Leica application suite software. The images collected were analyzed using the BD IPLAB imaging software and the data analysed using Graphpad Prism5 software. The error bars are the standard error of the mean (SEM). 24 images were used for each data point.

TMZ appeared to produce little reduction in growth of tumour cells in the 2D model compared to the untreated control cells. As the number of days in vitro increased the cells did not increase greatly in number (indicated by the average % fluorescence) until day 3 when there was a rapid rise in cell number that continued sharply to day 9 and then begins to level off to day 13. At concentrations of 1, 5 and 10 μM TMZ the growth curves were very similar to those of the control cells (FIG. 6a).

In the organotypic co-cultures (3D) there was a significant reduction in tumour cell growth below that of control cultures (as indicated by the average % fluorescence). All conditions showed an increase in cell number to day 3. The control cells and the 1 μM TMZ treated cultures continued to show an increase in cell number. At both 5 and 10 μM TMZ the cell number fell slightly from day 3 (FIG. 6b) with an IC 50 of ˜4 μM i.e. at 4 μM approximately half the cell growth had been inhibited (IC 50 inhibitory concentration for 50% decrease).

This could be linked to the changes in pH observed in Example 14. The drug TMZ is hydrolysed to active degradation products in a pH dependent manner and this may not occur when the tumour cells are cultured in isolation in a 2D system.

### Example 16

### Measurement of Response of Tumour Cells to Taxol. Comparison of the 3D Organotypic Tumour Co-Culture with 2D Monolayer Cultures of Tumour Cells

Paclitaxel (Taxol Sigma#T7402) has previously been shown to be toxic to glioblastoma cell lines in isolated 2D cultures, however, when used in clinical trials it has proved to have been ineffective. Using the same experimental conditions as in Example 14, the inventors have compared the cytotoxicity of taxol in the organotypic co-cultures (3D) and monolayer cultures of tumour cells alone (2D).

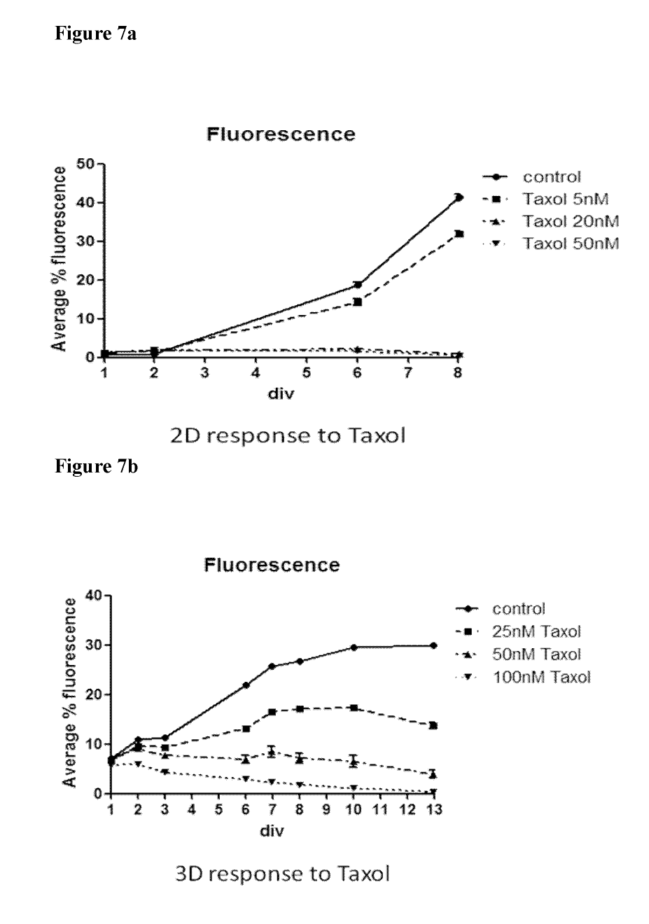

In monolayer cultures of tumour cells alone (2D) Control cultures grow slowly to day 2 then increase slowly over 8 days (as indicated by the average % fluorescence). This is seen with 5 nM taxol although there is a small but significant reduction in cell number. With 20 and 50 nM there is no increase in cell number throughout the experiment (FIG. 7a).

In the organotypic co-cultures (3D) there is a significant reduction in cell growth below that of control cultures (as indicated by the average % fluorescence) in all concentrations of taxol, 25, 50 and 100 nM. However, only 100 nM taxol reduces the cell number below base levels and the IC₅₀ was ˜25 nM (FIG. 7b). This IC₅₀ is at a much higher level than that required to completely kill all the cells in the 2D model.

This is probably due to the recruitment and activation of astrocytes in the organotypic co-cultures (3D) described in Example 12. There is evidence that astrocyte recruitment and activation is protective to glioblastoma from the action of Taxol.

### Example 17

### Measurement of Response of Tumour Cells to Cytosine Arabinoside (Ara-C): Comparison of the 3D Organotypic Tumour Co-Culture with 2D Monolayer Cultures of Tumour Cells

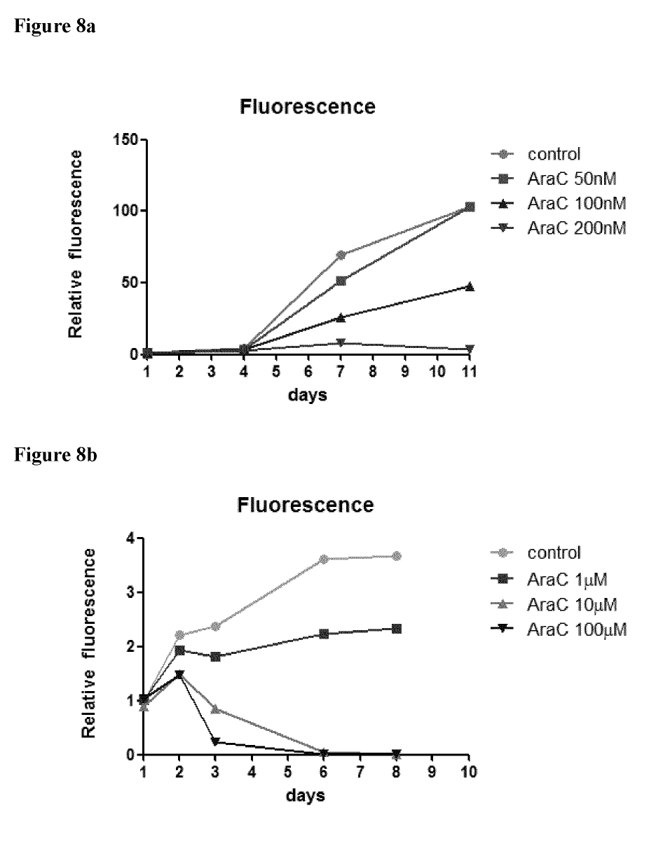

The inventors also tested Cytosine arabinoside (Ara-C), a further compounds that is known to inhibit proliferation of glioblastoma cell lines in 2D cultures but has failed in clinical trials: As shown in FIG. 8A, Ara-C showed anti-proliferative activity in 2D cultures (growth inhibition at 100 nM for AraC). When used on GBM/Brain co-cultures, 1 μM of Ara-C was required to reduce tumour cell proliferation, a dose ten times superior to the dose necessary in 2D cultures (FIG. 8B). At 10 μM, GBM cells were effectively killed.

### Example 18

### Effect of Taxol or Temozolomide on Tumours Formed from Various Cell Lines

Similar measurements were then performed with three other glioblastoma cell lines (GL-15, A172 and U87) growing in 2D or in co-cultures with brain cell matrices (3D) and treated with Taxol or Temozolomide. The growth inhibition IC50s were determined for each compound in each cell line and the results are summarized in table I. Overall, a decrease of activity of Taxol from 6 to 25 fold was observed in cells growing in co-cultures in comparison to 2D conventional cultures. Similarly to what has been observed with LN-18 cells, efficacy of Temozolomide was increased from 2.5 to 12 fold in brain-co-cultures. Thus, the co-cultures of the invention reflect the clinical situation more closely than 2D cultures for various glioblastoma cell lines.

TABLE 1

| | | IC₅₀ Taxol | | |
|---|---|---|---|---|
| Cell type | IC₅₀ Taxol 2D | 3D | IC₅₀ TMZ 2D | IC₅₀ TMZ 3D |
| LN-18 | ~7 nM | ~25 nM | >10 μM | ~4 μM |
| GL-15 | ~4 nM | 100 nM | >50 μM | ~20 μM |
| A172 | ~7 nM | ~50 nM | ~25 μM | ~5 μM |
| U87 | ~10 nM | ~60 nM | ~12 μM | ~1 μM |

### Example 19

### Characterisation of Tumour Regression in GBM/Brain Co-Cultures of the Invention

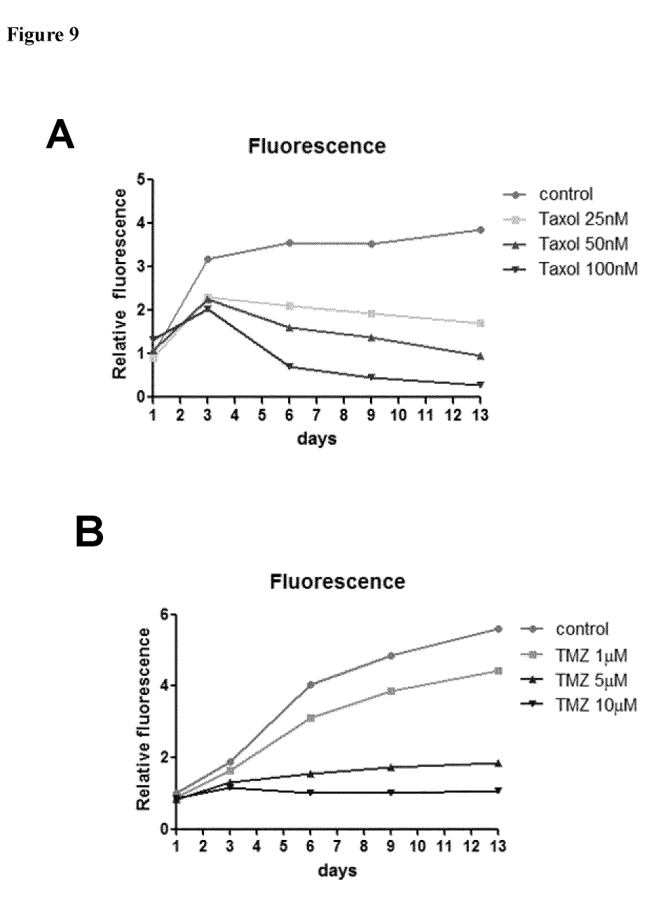

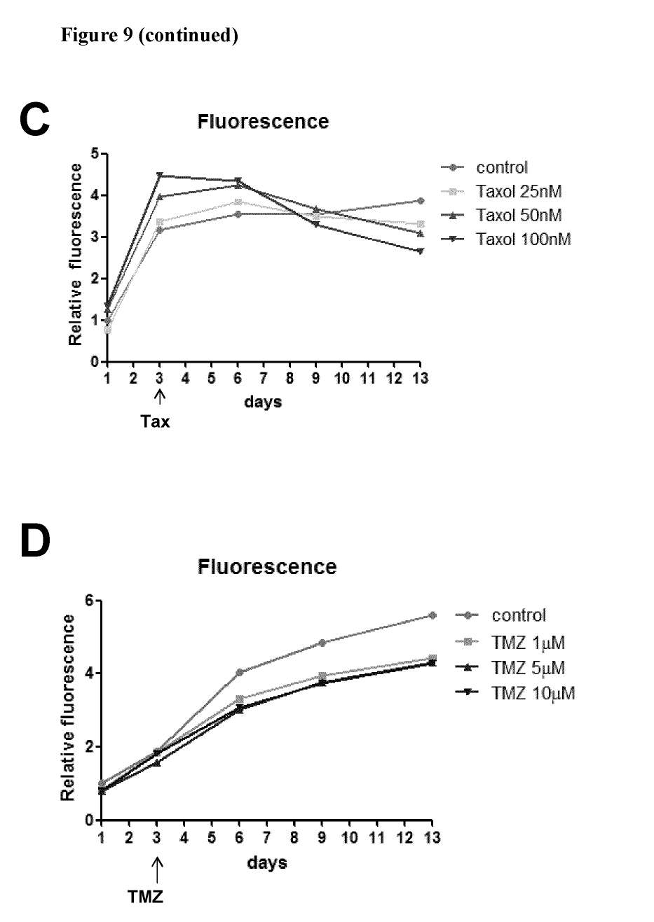

Chemotherapies are given to patients with established tumours and are poorly effective. To investigate whether Temozolomide or Taxol can cause tumour regression on established glioblastoma growing in co-cultures of the invention, the inventors started treatments either at the time of GBM/brain co-culture seeding (as above) or three days later, when tumour cell clusters are already present (FIG. 9). As observed above, when taxol treatment was started at the beginning of the experiment, growth rate of the tumour cell during the first three days was reduced. At 25 nM of taxol, cell growth was stopped and as observed before, 100 nM of taxol were required to achieve tumour clusters shrinkage.

When taxol treatment was started three days after GBM/brain co-culture seeding, when large tumour cell clusters are already formed, 25 nM of taxol had no effect on the fluorescence intensity, while 100 nM and 50 nM had marginal effects (compare FIGS. 9 A and C). The taxol concentrations required to achieve effective tumour shrinkage are very high in comparison to the concentrations that can be reached in human brain tumours and are far outside the therapeutic window. Interestingly, 5 or 10 μM of Temozolomide, two effective dose when used at cell seeding (FIGS. 6B and 9B), were poorly active on established GBM/brain co-cultures as they only slightly reduced tumour cell expansion (compare FIGS. 9B and 9D). Thus, also in terms of tumour regression, the properties of the co-cultures of the invention are representative of the clinical situation.

The experimental data given in the Examples above demonstrate advantageous properties of the 3D organotypic tumour co-cultures of the invention. In particular, tests of anti-tumour drugs and tumour-regression assays performed with these co-cultures more closely reflect the action of anti-tumour drugs in vivo than standard 2D models. The co-cultures of the invention are of high predictive value for studying the effect of potential anti-cancer agents, including for tumours which are strongly resistant to current therapies.

### REFERENCES

Beaupain, R., (1999) Methods in Cell Sciences, 21: 25-30
Becker-Hapak M. et al, (2001), Methods Vol 24 pp 247-56.
Damia, G., Maurizio D, Incalci, (2009) European Journal of Cancer, 45, 2768-2781
Kelland, L. R., (2004), European Journal of Cancer, 40, 827-836.
Muller et al (2001), Protocols for Neural Cell Culture, 3rd Ed. pp 13-2′7, S. Fedoroff and A. Richardson eds, Humana Press, Inc., Totowa, N.J. Interface Organotypic Hippocampal Slice Cultures.
Ridky et al. (2010) Nature Medicine 16(12):1450-1455)
Rygaard J, Povlsen C O., Acta Pathology Microbiol. Scand, 1969, 77, 758-760
Starzec et al (2003), Biology of Cells, 95, 257-264
Stoppini L., et al, (1991), Neurosci Methods, Vol 37 pp 173-82.
Vaira et al (2010), PNAS, 107:8352
van der Kuip et al (2006) BMC Cancer, 6:86
WO 2006/134432
WO 2006/136953

1.-36. (canceled)

37. An in vitro three-dimensional organotypic cell co-culture comprising tumour cells three-dimensionally disposed within a matrix of matrix cells which are distinct from the tumour cells, wherein the co-culture does not comprise a basement membrane.

38. The co-culture of claim 37, wherein the co-culture further comprises a semi-permeable support and a liquid culture medium, wherein
a) the co-culture is disposed upon a first surface of the support facing a gaseous phase;
b) a second surface of the support faces and is in contact with the liquid culture medium; and
c) the liquid culture medium is retained in contact with the support and with the culture by virtue of surface tension and/or capillarity.

39. The co-culture of claim 37, wherein the tumour cells are present in the form of aggregates of at least 5 cells.

40. A method of preparing a three-dimensional organotypic cell co-culture comprising the steps of
a) providing a first cell suspension comprising tumour cells and a second cell suspension comprising matrix cells which are distinct from the tumour cells;
b) combining the first and second cell suspensions to provide mixed cells suspended in a liquid suspension medium;
c) concentrating and/or compacting the mixed cells;
d) incubating the mixed cells obtained from step (c) on a first surface of a semi-permeable support facing a gaseous phase, wherein during the incubation a second surface of the support faces and is in contact with a liquid culture medium, and wherein the liquid is retained in contact with the support and the mixed cells by virtue of surface tension and/or capillary action.

41. The method of claim 40, wherein during the incubation of step (d), the tumour cells grow three-dimensionally disposed within a matrix formed by the matrix cells.

42. The method of claim 40, wherein providing the first and/or second cell suspensions comprises disassociating cells from a solid tissue source.

43. The method of claim 40, wherein the mixed cells prior to incubation comprise matrix and tumour cells in a matrix:tumour cell ratio from 100:1 to 10:1.

44. The method of claim 40, wherein the mixed cells are compacted in step (c) by capillary action, evaporation, centrifugation, gravitation, the application of hydrostatic pressure, pumping, suction, or aspiration.

45. The method of claim 40, wherein step (c) comprises centrifugation of the mixed cells and removal of supernatant liquid suspension medium.

46. The method of claim 45, wherein the mixed cells are placed upon the support of step (d) after the centrifugation and removal of supernatant liquid suspension medium of step (c).

47. The method of claim 45, wherein the mixed cells are placed upon the support of step (d) by the centrifugation.

48. The method of claim 40, wherein step (d) comprises allowing the cells to further compact upon the support.

49. The method of claim 48, wherein said further compaction is by evaporation and/or capillary action.

50. The method of claim 40, wherein the capillary action of step (d) further compacts the mixed cells.

51. The method of claim 40, wherein the capillary action of step (d) also compacts the mixed cells in step (c).

52. The method of claim 40, wherein the method further comprises the step of cryopreserving the organotypic co-culture.

53. The co-culture of claim 38, wherein the second surface of the support is contralateral to the first surface.

54. The co-culture of claim 37, wherein the matrix cells are non-cancerous cells.

55. The co-culture of claim 37, wherein the matrix cells comprise more than one cell type.

56. The co-culture of claim 37, wherein the matrix cells consist of a single cell type.

57. The co-culture of claim 37, wherein the tumour cells are not genetically engineered.

58. The co-culture of claim 37, wherein the tumour and/or matrix cells are mammalian.

59. The co-culture of claim 37, wherein the tumour and/or matrix cells are human.

60. The co-culture of claim 37, wherein the matrix cells comprise stem cells, primary cells, stromal cells, or cells derived from a source selected from a biopsy, cadavaric tissue, mammalian or human tissue-derived cell lines, the central nervous system, brain, bone marrow, blood, spleen, retina thymus, heart, mammary gland, liver, pancreas, thyroid, skeletal muscle, kidney, lung, intestine, ovary, bladder, testis, uterus or connective tissue.

61. The co-culture of claim 37, wherein the tumour cells are cancer stem cells

62. The co-culture of claim 37, wherein the tumour cells are derived from a tumour of stem cells, pancreas, blood, cervix, colon, intestine, kidney, brain, mammary gland, ovary, prostate, skin, liver, lung or spleen.

63. The co-culture of claim 38, wherein the support comprises hydrophilic PTFE, PET or aluminium oxide.

64. The co-culture of claim 38, wherein the support is optically transparent.

65. The co-culture of claim 38, wherein the support is coated with a material that facilitates adhesion of the co-culture to the support.

66. The co-culture of claim 38, wherein the material coating the support is selected from collagen, laminin or fibronectin.

67. An in vitro three-dimensional organotypic cell co-culture obtainable by the method of claim 40.

68. A method for screening for anti-cancer agents, said method comprising contacting the co-culture of claim 37 with a test agent, wherein an inhibitory effect of the test agent upon the growth, proliferation, viability or migration of the tumour cells of the co-culture indicates that the test agent is a candidate anti-cancer agent.

69. An assay kit comprising one or more co-cultures of claim 37.

70. A device for screening compounds comprising one or more co-cultures of claim 37.
